(19) United States
(12) Patent Application Publication
(10) Pub. No.: US 2022/0047420 A1
(43) Pub. Date: Feb. 17, 2022

(54) SYSTEMS AND METHODS FOR DRUG DELIVERY TO OCULAR TISSUE

(71) Applicant: Regeneron Pharmaceuticals, Inc., Tarrytown, NY (US)

(72) Inventors: Bryan GRYGUS, Clifton Park, NY (US); Matthew PAUSLEY, Gansevoort, NY (US); Megan DeRIDDER, Gansevoort, NY (US); Paige WAECHTER, Cohoes, NY (US); Prithvi SINGH, Cohoes, NY (US); Ross KENYON, Saratoga Springs, NY (US); Sibgat ULLA, Rensselaer, NY (US); Trevor LANGLEY, Rensselaer, NY (US); Bibiana IGLESIAS, Hudson, NY (US)

(73) Assignee: Regeneron Pharmaceuticals, Inc., Tarrytown, NY (US)

(21) Appl. No.: 17/444,897

(22) Filed: Aug. 11, 2021

Related U.S. Application Data
(60) Provisional application No. 63/064,658, filed on Aug. 12, 2020.

(51) Int. Cl. A61F 9/00

(52) U.S. Cl. CPC A61F 9/0017; A61B 2017/3405; A61F 9/0026

(57) ABSTRACT

According to one aspect of the disclosure, an apparatus for manipulating a sclera to facilitate delivery of a medicament to a suprachoroidal space of an eye may include a needle with a sharp distalmost tip, a needle hub connected to a proximal end of the needle, a housing surrounding the needle hub and extending from a proximal end of the needle hub, and an adaptor surrounding a portion of the needle.

### CROSS-REFERENCE TO RELATED APPLICATIONS

This application claims priority to U.S. Provisional Application No. 63/064,658, filed on Aug. 12, 2020, the entirety of which is incorporated by reference herein.

### TECHNICAL FIELD

Various aspects of the present disclosure relate generally to delivering drugs to ocular tissue. More specifically, the present disclosure relates to instruments and related methods for delivering drugs to the suprachoroidal space of an eye.

### INTRODUCTION

Eye conditions and diseases lead to optic nerve damage and visual field loss. Medications, laser surgery, and/or incisional surgery are interventions that may be employed to help lower intraocular pressure, save the subject's existing vision, and delay further progression of the condition and/or disease. With respect to incisional surgery, instruments for performing surgical procedures, devices for delivery drug therapies, and methods made possible by such instruments, are highly sought after to provide improved outcomes for users and subjects.

### SUMMARY OF THE DISCLOSURE

According to one aspect of the disclosure, is a system for delivering medicament to a suprachoroidal space of an eye comprising a needle having a passage therethrough and a sharp distalmost tip and an apparatus configured to manipulate the sclera to facilitate delivery of the medicament to the suprachoroidal space of the eye.

Various embodiments of the system may include one or more of the following aspects: the needle may be configured to deliver the medicament to the suprachoroidal space of the eye; the sharp distalmost tip may include a plurality of openings, wherein the openings may include a circular configuration or slots, or wherein the plurality of openings are present on at least a portion of a circumference and a length of the sharp distalmost tip; the apparatus may be configured to deliver the medicament to the suprachoroidal space of the eye; the apparatus may be disposed within the passage of the needle and longitudinally translatable relative to the needle; the apparatus may include a tubular shaft having a distal end, wherein the tubular shaft comprises a rigid material, a semi-rigid material, or a flexible material; the distal end may include an atraumatic distal tip, wherein the atraumatic distal tip may include a plurality of openings, further wherein the openings may include a circular configuration or slots, or wherein the plurality of openings are present on at least a portion of a circumference and a length of the atraumatic distal tip; the distal end may include an expandable member, wherein the expandable member may include a stent; the tubular shaft may include an expandable portion, wherein the expandable portion may include a pair of curved arms positioned proximally of the atraumatic distal tip; the tubular shaft may include a cross-sectional dimension less than a cross-sectional dimension of the passage; an outer surface of the tubular shaft may include one or more channels; the distal end of the tubular shaft may include at least two legs configured to selectively diverge when the distal end of tubular shaft is deployed from the passage of the needle; the needle may be curved; the needle may be U-shaped; the needle may include a bend positioned proximally of the sharp distalmost tip; the needle may include an outer surface having a plurality of geometric features configured to provide tactile feedback to a user; or the distal end may include an anchor, wherein the anchor may have a curved, planar, or atraumatic shape.

In another aspect, a system of the present disclosure may comprise a planar surface having a projection for deforming one or more of the sclera or choroid, wherein the projection may be a rounded surface. In another aspect, a system of the present disclosure may include a chamber configured to draw a portion of the sclera therein, wherein the apparatus may be configured to apply a suction to the sclera; the needle may be disposed within the chamber; the chamber may include a stop configured to limit proximal advancement of the sclera into the chamber; the stop may be configured to surround the needle; the stop may include a plurality of extensions extending from a sidewall of the chamber towards a center of the chamber; the stop may include a plurality of openings configured to facilitate application of suction to the sclera; the chamber may include a circular cross-section configuration; or the chamber may include a semi-circular cross-sectional configuration.

The present disclosure includes an apparatus for manipulating a sclera to facilitate delivery of a medicament to a suprachoroidal space of an eye, the apparatus comprising a tubular shaft having a distal end, wherein the tubular shaft comprises a rigid, semi-rigid, or flexible material; and a needle having a passage therethrough and a sharp distalmost tip, wherein the apparatus is disposed within the needle. Various embodiments of the apparatus may include one or more of the following aspects: the needle may be configured to deliver the medicament to the suprachoroidal space of the eye; the sharp distalmost tip may include a plurality of openings, wherein the openings may include a circular configuration or slots, or wherein the plurality of openings are present on at least a portion of a circumference and a length of the sharp distalmost tip; the apparatus may be configured to deliver the medicament to the suprachoroidal space of the eye; the apparatus may be longitudinally translatable relative to the needle; the distal end may include an atraumatic distal tip, wherein the atraumatic distal tip may include a plurality of openings, wherein the openings may include a circular configuration or slots, or wherein the plurality of openings are present on at least a portion of a circumference and a length of the atraumatic distal tip; the distal end may include an expandable member, wherein the expandable member may include a stent; the tubular shaft may include an expandable portion wherein the expandable portion may include a pair of curved arms positioned proximally of the distal end; the tubular shaft may include a cross-sectional dimension less than a cross-sectional dimension of the passage; an outer surface of the tubular shaft may include one or more channels; the distal end of the tubular shaft may include at least two legs configured to selectively diverge when the distal end of tubular shaft is deployed from the passage of the needle; the needle may be curved; or the distal end may include an anchor, wherein the anchor has a curved, planar, or atraumatic shape.

In another aspect, the present disclosure includes an apparatus for manipulating a sclera to facilitate delivery of a medicament to a suprachoroidal space of an eye, the apparatus comprising a planar surface having a projection for deforming one or more of the sclera or choroid, wherein the projection may be a rounded surface.

In another aspect, the present disclosure includes an apparatus for manipulating a sclera to facilitate delivery of a medicament to a suprachoroidal space of an eye, the apparatus comprising a chamber configured to draw a portion of the sclera therein. Various embodiments of the apparatus may include one or more of the following aspects: the apparatus may be configured to apply a suction to the sclera; a needle may be disposed within the chamber; the chamber may include a stop configured to proximal advancement of the sclera into the chamber; the stop may be configured to surround the needle; the stop may include a plurality of extensions extending from a sidewall of the chamber towards a center of the chamber; the stop may include a plurality of openings configured to facilitate application of suction to the sclera; the chamber may include a circular cross-section configuration; or the chamber may include a semi-circular cross-sectional configuration.

In another aspect, the present disclosure includes an apparatus for manipulating a sclera to facilitate delivery of a medicament to a suprachoroidal space of an eye, the apparatus comprising a needle tubing having a cylindrical shape and a serrated needle tip, wherein the serrated needle may oscillate to cut through a portion of the sclera.

In another aspect, the present disclosure includes an apparatus for manipulating a sclera to facilitate delivery of a medicament to a suprachoroidal space of an eye, the apparatus comprising a needle with a sharp distalmost tip; a needle hub connected to a proximal end of the needle; and a housing surrounding the needle hub and extending from a proximal end of the needle hub. Various embodiments of the apparatus may include one or more of the following aspects: the housing may be cylindrical shaped; the housing may comprise an additional component chosen from a syringe, a spring, a piston, a plunger rod, an indicator, a feedback mechanism, or combinations thereof; or a shaft surrounding a portion of the needle and extending from a distal end of the needle hub, wherein a distal end of the shaft is angled, allowing for insertion of the needle at an angle.

In another aspect, the present disclosure is drawn to a method for delivering a medicament to a suprachoroidal space of an eye, the method comprising manipulating one of a sclera and a choroid layer of the eye to increase a dimension of the suprachoroidal space; advancing a distal end of a medicament delivery device to the suprachoroidal space; positioning a distalmost tip of the medicament delivery device in the suprachoroidal space; and delivering a volume of the medicament to the suprachoroidal space. Various embodiments of the method may include one or more of the following aspects: advancing the distal end of the medicament delivery device to the suprachoroidal space may include penetrating the sclera; positioning the distalmost tip of the medicament delivery device may include disposing the distalmost tip into the suprachoroidal space without contacting the choroid; positioning the distalmost tip of the medicament delivery device may include disposing the distalmost tip into the suprachoroidal space without piercing an outermost surface of the choroid; positioning the distalmost tip of the medicament may include disposing the distalmost tip in the suprachoroidal space without penetrating a thickness of the choroid; a volume of the medicament delivered to the suprachoroidal space may be approximately 50 uL to 500 uL; delivery of the volume of the medicament to the suprachoroidal space may be pressure controlled; manipulating one of the sclera and the choroid layer may include rotating the medicament delivery device; manipulating one of the sclera and choroid layer may include pulling the sclera layer to increase the dimension of the suprachoroidal space; delivering the volume of the medicament to the suprachoroidal space may include delivering the volume from the distalmost tip of the medicament delivery device; or delivering a volume of the medicament to the suprachoroidal space may include delivering the medicament from a location proximally of the distalmost tip of the medicament delivery device.

In another aspect, the present disclosure includes an apparatus to facilitate directed delivery of a medicament into a human organ of a patient. The apparatus may include a container for a medicament fluidly connected to a needle, the needle comprising a needle shaft and a sharp distalmost tip with a bevel, wherein the needle is connected to a distal end of the container, and an adaptor surrounding a portion of the needle shaft along its longitudinal axis, but not including the sharp distalmost tip of the needle, the adaptor including an outermost slanted surface configured to direct a trajectory of the sharp distalmost tip to a pre-determined depth and location within the human organ, wherein the outermost slanted surface faces an identical direction as the bevel of the sharp distalmost tip, wherein an angle of the outermost slanted surface dictates the trajectory of the needle, and wherein a length of the needle extending from the outermost slanted surface determines the depth and location of the medicament delivery. Various embodiments of the apparatus may include one or more of the following aspects: the sharp distalmost tip may be a portion of the needle extending from a distal end of the adaptor; a needle hub shaft connected to a proximal end of the needle, such as a staked needle; a hub disposed between the container and the needle; the needle is removably connected to the hub; the needle is a first needle, and the apparatus further comprises a second needle; the first needle and the second needle are interchangeable; the needle is replaceable; the angle ranges from about 25 degrees to about 75 degrees; the angle ranges from about 40 degrees to about 60 degrees; the angle is about 45 degrees; the adaptor is connected to a portion of the needle shaft via a fastener or screw; the adaptor is translatable relative to an axial path of the needle shaft; the adaptor is attached to the needle shaft via an adhesive; the adaptor may include: a proximal end having a surface extending in a first plane perpendicular to the needle, an angled distal side, an intermediate surface extending between the proximal end and the angled distal side, wherein the intermediate surface extends in a second plane perpendicular to the first plane, and a distal end, wherein the distal end includes a substantially flat surface extending in a third plane parallel to the first plane; at least a portion of the adaptor includes a substantially cylindrically shaped cross-section; the outermost slanted surface is angled relative to a longitudinal axis of the adaptor; a portion of the sharp distalmost tip of the needle extends beyond a distal end of the adaptor; the sharp distalmost tip of the needle has a length ranging from about 600 μm to about 800 μm; the outermost slanted surface is a planar surface; and the outermost slanted surface is a convex surface configured to mate with an outer surface of an eye.

In another aspect, the present disclosure includes a system for delivering medicament to an ocular space of a patient. The system may include a syringe with a nominal maximum fill volume of between about 0.5 mL and about 1.0 mL; a needle comprising a needle shaft and a sharp distalmost tip with a bevel; and an adaptor surrounding a portion of the needle shaft along its longitudinal axis, the adaptor including an outermost slanted surface angled relative to a longitudinal axis of the adaptor and wherein the adaptor is configured to direct a trajectory of the sharp distalmost tip to a pre-determined ocular depth and location; wherein the syringe, needle, and adaptor are sterilized and contained in a blister pack. Various embodiments of the system may further include one or more of the following aspects: the adaptor is configured to limit advancement of the distalmost tip into the suprachoroidal space of the eye; the sharp distalmost tip is a portion of the needle extending from the outermost slanted surface; an angle of the outermost slanted surface ranges from about 25 degrees to about 75 degrees; an angle of the outermost slanted surface ranges from about 40 degrees to about 60 degrees; an angle of the outermost slanted surface is about 45 degrees; and the sharp distalmost tip of the needle has a length ranging from about 600 μm to about 800 μm.

In another aspect, the present disclosure includes a kit for treating a patient suffering from an ocular disease. The kit may include: a syringe with a nominal maximum fill volume of between about 0.5 ml and about 1.0 ml; a needle having a needle shaft and a sharp distalmost tip; an adaptor surrounding a portion of the needle shaft along its longitudinal axis, but not including the sharp distalmost tip of the needle, the adaptor including an outermost slanted surface configured to direct the trajectory of the sharp distalmost tip to a pre-determined ocular depth and location; and an ophthalmic drug.

In another aspect, the present disclosure includes a kit for treating a patient suffering from an ocular disease. The kit may include: a syringe pre-filled with an ophthalmic drug, wherein a volume of the ophthalmic drug ranges between about 0.5 ml and about 1.0 ml; a needle having a needle shaft, a passage therethrough, and a sharp distalmost tip; and an adaptor surrounding a portion of the needle shaft along a longitudinal axis of the needle shaft, the portion excluding the sharp distalmost tip of the needle, the adaptor including an outermost slanted surface configured to direct a trajectory of the sharp distalmost tip to a predetermined ocular depth and location.

In another aspect, the present disclosure includes a method of delivering a medicament to an eye of a patient. The method may include: positioning a distalmost tip of a medicament delivery device in a suprachoroidal space of the eye at a predetermined ocular depth and location, wherein the medicament delivery device may comprise: a container for a medicament fluidly connected to a needle, the needle comprising a needle shaft and a sharp distalmost tip with a bevel, wherein the needle is connected to a distal end of the container, and an adaptor surrounding a portion of the needle shaft along its longitudinal axis, the portion not including the sharp distalmost tip of the needle, the adaptor including an outermost slanted surface configured to direct the trajectory of the sharp distalmost tip to a pre-determined ocular depth and location; and delivering a volume of the medicament to the suprachoroidal space.

In another aspect, the present disclosure includes a method of delivering a medicament to a suprachoroidal space of an eye using a medicament device. The medicament device may include: a container for a medicament fluidly connected to a needle, the needle comprising a needle shaft and a sharp distalmost tip with a bevel; and an adaptor surrounding a portion of the needle shaft along its longitudinal axis, the adaptor including an outermost slanted surface angled with respect to the longitudinal axis. The method may include: penetrating a sclera of the eye with the sharp distalmost tip; inserting the needle through the sclera into the suprachoroidal space until the outermost slanted surface contacts the sclera; and upon the outermost slanted surface contacting the sclera, delivering a volume of the medicament to the suprachoroidal space.

In another aspect, the present disclosure includes a method of delivering a medicament to a suprachoroidal space of an eye. The method may include: manipulating one of a sclera or a choroid layer of the eye to increase a dimension of the suprachoroidal space; advancing a distal end of a medicament delivery device to the suprachoroidal space; positioning a distalmost tip of the medicament delivery device in the suprachoroidal space; and delivering a volume of the medicament to the suprachoroidal space. Various embodiments of the method may further include one or more of the following aspects: advancing the distal end of the medicament delivery device to the suprachoroidal space includes penetrating the sclera; positioning the distalmost tip of the medicament delivery device includes disposing the distalmost tip into the suprachoroidal space without contacting the choroid; positioning the distalmost tip of the medicament delivery device includes disposing the distalmost tip into the suprachoroidal space without piercing an outermost surface of the choroid; positioning the distalmost tip of the medicament includes disposing the distalmost tip in the suprachoroidal space without penetrating a thickness of the choroid; and a volume of the medicament delivered to the suprachoroidal space is approximately 50 uL to 500 uL.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIGS. 1A and 1B are cross-sectional views of an exemplary instrument treating ocular tissue, according to an embodiment of the present disclosure.

FIG. 2 is a cross-sectional view of an exemplary instrument treating ocular tissue, according to an embodiment of the present disclosure.

FIGS. 3A and 3B are cross-sectional views of an exemplary instrument, according to an embodiment of the present disclosure.

FIGS. 4A and 4B are cross-sectional views of an exemplary instrument treating ocular tissue, according to an embodiment of the present disclosure.

FIGS. 5A and 5B are cross-sectional views of an exemplary instrument, according to an embodiment of the present disclosure.

FIG. 6A is a cross-sectional view of an exemplary instrument, and FIG. 6B is a top view of the FIG. 6A instrument, according to an embodiment of the present disclosure.

FIGS. 7A-7C depict an exemplary instrument treating ocular tissue, according to an embodiment of the present disclosure.

FIGS. 8A-8F depict another exemplary instrument treating ocular tissue, according to an embodiment of the present disclosure.

FIG. 9 depicts another exemplary instrument treating ocular tissue, according to an embodiment of the present disclosure.

FIG. 10 depicts a further exemplary instrument treating ocular tissue, according to an embodiment of the present disclosure.

FIGS. 11A and 11B depict another exemplary instrument, according to an embodiment of the present disclosure.

FIGS. 12A and 12B are cross-sectional views of an exemplary tubular shaft of an exemplary instrument, according to an embodiment of the present disclosure.

FIG. 13 is a side view of another exemplary instrument, according to an embodiment of the present disclosure.

FIGS. 14-19 are cross-sectional views of a yet further exemplary instrument treating ocular tissue, according to an embodiment of the present disclosure.

FIG. 20 is a top view of an exemplary instrument treating ocular tissue, according to an embodiment of the present disclosure.

FIGS. 21A and 21B are perspective views of another exemplary instrument, according to an embodiment of the present disclosure.

FIGS. 22A and 22B are perspective views of a further exemplary instrument, according to an embodiment of the present disclosure.

FIGS. 23A and 23C are cross-sectional views of yet another exemplary instrument, according to an embodiment of the present disclosure. FIG. 23B is a cross-section view showing the exemplary instrument of FIGS. 23A and 23C treating ocular tissue, according to an embodiment of the present disclosure.

FIG. 24 is a partially-transparent view of an exemplary instrument, according to an embodiment of the present disclosure.

FIG. 25 is a partially-transparent view of another exemplary instrument, according to an embodiment of the present disclosure.

FIGS. 26A and 26B are partially-transparent views of an exemplary instrument, according to an embodiment of the present disclosure.

FIGS. 27A and 27B are cross-sectional views showing a further embodiment of an instrument being used to deliver a drug to ocular tissue, according to an embodiment of the present disclosure.

FIG. 28 is a perspective view of an exemplary instrument, according to an embodiment of the present disclosure.

FIG. 29 is a cross-sectional view of an exemplary instrument, according to an embodiment of the present disclosure.

FIG. 30 depicts an exemplary instrument treating ocular tissue, according to an embodiment of the present disclosure.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% in a stated numeric value.

Aspects of the disclosure relate to, among other things, instruments and methods for delivering drugs to ocular tissues. Each of the aspects disclosed herein may include one or more of the features described in connection with any of the other disclosed aspects. It may be understood that both the foregoing general description and the following detailed description are exemplary and explanatory only and are not restrictive of any claimed inventions.

The suprachoroidal space (SCS) is a potential space between the sclera and choroid that traverses the circumference of the posterior segment of the eye. The SCS is a useful site for drug delivery because it targets the choroid, retinal pigment epithelium, and retina with high bioavailability, while maintaining low levels elsewhere in the eye. Under physiological conditions, primarily due to intraocular pressure (IOP), the SCS is primarily in a collapsed state. The SCS plays a role in maintaining IOP via uveoscleral outflow, which is an alternative drainage route for the aqueous humor, and is a natural flow path from the front to the back of the eye. Due to its role in maintaining IOP, the SCS has the potential to expand and contract in response to the presence of fluid. The SCS may expand to accommodate different volumes, for example, up to about 3.0 mm, depending on injection volumes. Injecting high volumes of drugs may have adverse effects, for example, elevated IOP, which may cause localized serous retinal elevation, choroidal hemorrhage away from needle entry, and choroidal edema and potential choroidal detachment; backflow from needle entry; and reflux of fluid which may cause subconjunctival hemorrhage. Additionally high volumes of fluid may not be injected into the eye until a needle of an injection device has fully penetrated the sclera.

To expand the SCS, e.g., by separating the sclera and choroid mechanically and breaking down fibers holding the sclera and choroid together, instruments may be inserted through the sclera and placed at the correct depth between the sclera and choroid layers, such that optimal volumes of fluids, e.g., drugs, may be injected into the SCS. Any drugs inserted into the SCS may allow for direct drug delivery to the posterior section of the eye to specifically target, e.g., the retina and/or macula. Instruments and methods for insertion and injection into the eye may only allow for extension into a certain depth of the ocular layers. For example, the sclera layer ranges from about 500 μm to about 1100 μm, the SCS has a thickness of about 35 μm, and the choroid layer ranges from about 50 μm to about 300 μm. Depth of insertion of an instrument for drug delivery into the ocular layers may range from about 1 mm to about 10 mm. However, such a depth of insertion may penetrate and/or impact additional layers of the ocular tissue, e.g., the choroid, retinal pigment epithelium (RPE), and retina. Penetration of such layers should be minimized as much as possible, such that the desired drug may be directed into the targeted area of the eye via a minimally invasive procedure. For example, injection procedures may be performed as an outpatient procedure. Instruments and methods discussed in the present disclosure address the disadvantages described above, and may increase the ability of the SCS to hold and diffuse optimal volumes of drugs, for example, 50 μL to 500 μL.

The example embodiments described herein may be used in the treatment of a variety of conditions, including ocular conditions. For example, embodiments of the present disclosure may be used in the treatment of refractive errors, macular degeneration, cataracts, retinopathy, retinal detachments, glaucoma, amblyopia, strabismus, any other ocular condition, or any other condition suitable for treatment via tissue in the eye.

The description above and examples are illustrative, and are not intended to be restrictive. One of ordinary skill in the art may make numerous modifications and/or changes without departing from the general scope of the invention. For example, and as has been referenced, aspects of above-described embodiments may be used in any suitable combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description.

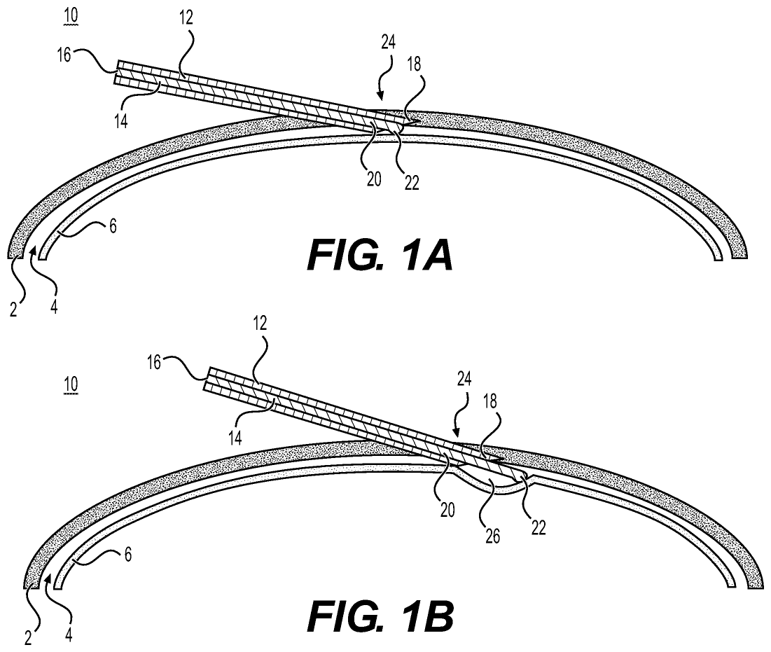

Referring to FIGS. 1A and 1B, the different tissues and layers of the eye are represented, e.g., sclera 2, SCS 4, and choroid 6. FIGS. 1A-4B show various views of an instrument 10 for manipulating layers of ocular tissue to facilitate delivery of a medicament to a suprachoroidal space of an eye. Instrument 10 may include a needle 12 and tubular shaft 14. Needle 12 may have a passage 16 and a distalmost tip 18. Tubular shaft 14 may be disposed in needle 12, and may be longitudinally translatable relative to needle 12, such that instrument 10 may be inserted into a subject's eye to create a separation between different tissues in the eye (e.g., by moving the tissues away from each other, breaking down fibers and/or bonds in the different tissues) and/or remove tissue within the eye, while at least a portion of instrument 10 may remain outside of the eye where it may be held by a user. A medicament, i.e., drug, may be contained within needle 12, tubular shaft 14, or both needle 12 and tubular shaft 14. In examples wherein instrument 10 may be inserted into a subject's eye and tubular shaft 14 may be positioned to create and/or increase SCS 4 (FIG. 1B), the medicament may more easily flow away from an instrument insertion site 24 and spread over an area of the tissues and/or an expanded area 26 of SCS 4 to help prevent adverse reactions associated with drug injection. Spreading of the medicament over the tissue area may also prevent backflow of the medicament from instrument insertion site 24 and improve bioavailability to a posterior section of the eye.

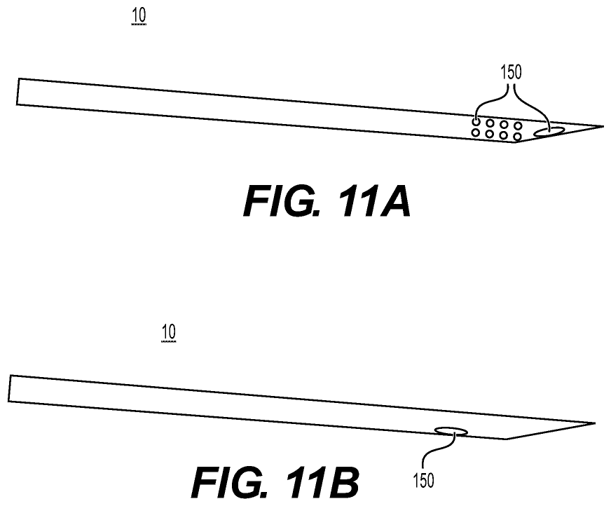

Distalmost tip 18 may be a sharp tip or needle configured to penetrate a tissue layer of the eye, e.g., sclera 2. Distal end 20 may have a substantially atraumatic or blunt tip 22 resistant to penetration of choroid 6 (FIG. 1A). Once instrument 10 is inserted into a subject's eye, tubular shaft 14 may be longitudinally pushed through passage 16 to separate sclera 2 and choroid 6 (e.g., by pushing and/or otherwise deforming the sclera 2 and/or choroid 6), which may form an expanded portion 26 of SCS 4 (FIG. 1B). Instrument 10 may include a plurality of openings 150 allowing a drug to flow from instrument 10 into SCS 4. For example, openings 150 may be circular, slots, or combinations thereof (FIGS. 11A and 11B). Openings 150 may be arranged on needle 12, tubular shaft 14, or both needle 12 and tubular shaft 14, in any appropriate configuration to allow a drug to flow from instrument 10. For example, openings 150 may be positioned along a length of and/or radially around a circumference of needle 12 (e.g., in a sidewall of needle 12), tubular shaft 14, or both needle 12 and tubular shaft 14. In some examples, instrument 10 may include from 2 to 30 openings, or more than 30 openings. Referring to FIGS. 11A and 11B, instrument 10 may be inserted into a patient's eye, parallel to the plane of SCS 4, such that a drug may flow out of instrument 10 over an area of SCS 4 to help prevent adverse reactions associated with a drug into SCS 4.

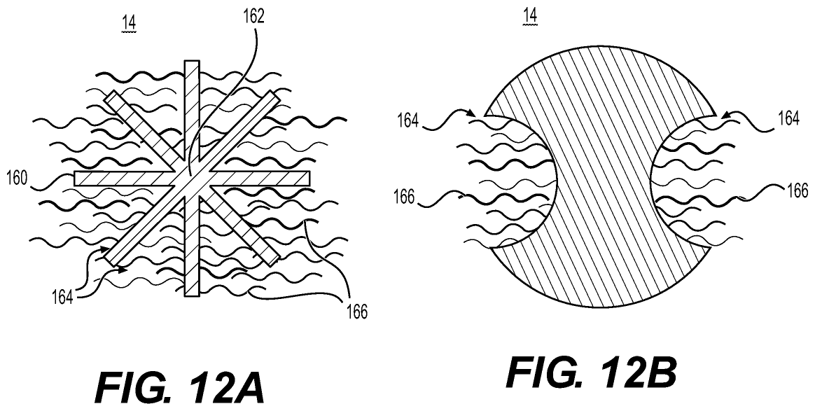

Tubular shaft 14 may be solid, i.e., containing no opening, such that a drug may flow out of needle 12 and flow around tubular shaft 14. In such example, tubular shaft 14 may include a cross-section dimension less than a cross-section dimension of passage 16, so that the drug may flow around tubular shaft 14. Tubular shaft 14 may have various configurations to facilitate drug flow around tubular shaft 14 and away from insertion site 24 (FIG. 1B). FIGS. 12A and 12B show exemplary geometries of tubular shaft 14. For example, tubular shaft 14 may have a star shape (FIG. 12A) with a plurality of arms 160 extending from and connected to a radial center 162 allowing tubular shaft 14 containing channels 164 to gently separate sclera 2 and choroid 6. The channels 164 may allow a drug to flow out of needle 12, around tubular shaft 14, and away from injection site 24. The drug flow 166 around arms 160 of tubular shaft 14 is shown in FIG. 12A. In another example, tubular shaft 14 may include at least a pair of channels 164 (FIG. 12B), such that needle 12 may be inserted in a specific orientation so that channels 164 are oriented within the plane of SCS 4. When a drug is injected through needle 12, the drug may flow through channels 164 and be pushed outward/away from tubular shaft 14. Such configurations of tubular shaft 14 may be beneficial during the manufacturing process of instrument 10, as channels 164 may be formed on an exterior surface of tubular shaft 14, as opposed to an interior area of tubular shaft 14. The drug flow 166 through channels 164 of tubular shaft 14 is shown in FIG. 12B. Such configurations of tubular shaft 14 may further be useful for injecting substances of differing viscosities into SCS 4. For example, a low viscosity fluid may be injected through channels 164, which advantageously cause the low viscosity fluid to spread out across in SCS 4. Tubular shaft 14 may subsequently be removed from needle 12, effectively increasing the size of the flow path. A viscous fluid, such as a gel, may then be injected through needle 12. Tubular shaft 14 may therefore both promote diffusion of lower viscosity fluids without permanently obstructing the flow path for higher viscosity fluids.

In still another example, different substances may be caused to flow through each of the channels 164 of tubular shaft 14. For example, a drug may be caused to flow through one of the channels 164 and another substance may be caused to flow through another of the channels 164. The substances may be caused to flow through the channels 164 and injected into the patient's eye either sequentially or in parallel. Such a configuration may be useful in situations, for example, in which a drug is in pellet form and requires hydration to be released from the pellets into tissue. The drug pellets may be injected through one of the channels 164 and a hydrating fluid may be caused to flow through another of the channels 164. Upon exiting the channels 164, the pellets and hydrating fluid may mix to allow the drug to be released from the pellets into tissue. As another example, substances of varying viscosities may be injected into the eye, each through separate channels 164. As still another example, substances that polymerize when mixed may be injected into the eye, each through separate channels 164. By injecting the substances through separate channels 164, separation of the substances may be maintained and polymerization may be prevented until the substances enter the target space of the eye.

In some embodiments, tubular shaft 14 may be formed entirely or partially of an absorbent material, such as a sponge, for example. In such configurations, tubular shaft 14 may be used to absorb fluid that accumulates in SCS 4 or in other portions of a patient's eye. Fluid accumulation may occur due to insertion of instrument 10 into a patient's eye, hemorrhaging, bleeding, or general build-up. Upon insertion of instrument 10 into SCS 4, tubular shaft 14 may be selectively translated relative to needle 12 toward SCS 4 to absorb fluid located near distalmost tip 18 in SCS 4. In embodiments in which tubular shaft 14 is formed partially of an absorbent material, blunt tip 22 (as shown in FIGS. 1A and 1B) of tubular shaft 14 may be formed of the absorbent material. Blunt tip 22 may itself similarly be formed either entirely or partially of the absorbent material.

In some embodiments, tubular shaft 14 may alternatively be formed of a drug or medicament. For example, tubular shaft 14 may be formed of a solidified drug, such as a lyophilized drug. Upon insertion of instrument 10 into SCS 4, tubular shaft 14 may be translated relative to needle 12 toward SCS 4. When tubular shaft 14 is extended into SCS 4, tubular shaft 14, or a portion thereof, may break off or otherwise disconnect from instrument 10, thereby permitting application of the drug into SCS 4.

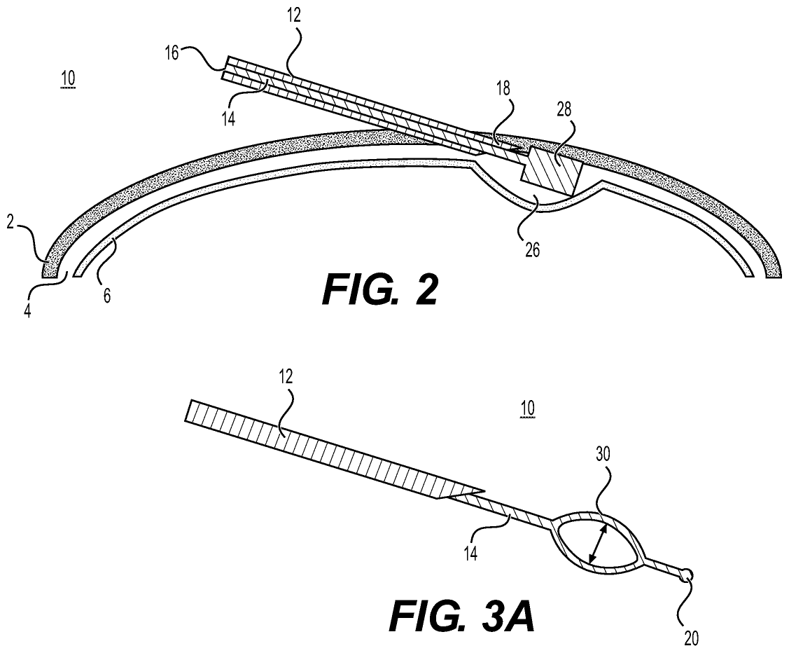

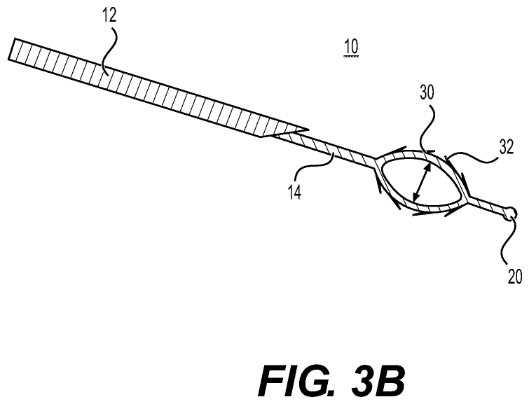

Distal end 20 may include an expandable member 28 (FIG. 2). Expandable member 28 may be any appropriate component, for example, a stent or a balloon. Expandable member 28 may expand in a vertical direction to separate sclera 2 and choroid 6 to increase a thickness of SCS 4 and/or a horizontal direction to expand over an area of SCS 4. For example, expandable member 28 may expand sclera 2 in an upwards direction, i.e., away from choroid 6, to increase SCS 4. In other words, expandable member 28 may expand to allow an increased amount of a drug to more easily flow from instrument 10 into SCS 4. Referring to FIG. 3A, tubular shaft 14 may include an expandable portion 30. Expandable portion 30 may expand in a vertical direction and/or a horizontal direction to expand over an area of SCS 4. For example, expandable portion 30 may include a pair of curved arms positioned proximally of distal tip 20 (FIG. 3A), such that a drug may flow out of expandable portion 30 from injection site 24 and into an expanded area 26 of SCS 4. In some examples, the drug may flow out of a plurality of openings 150 on tubular shaft 14 and/or expandable portion 30. In other examples, expandable portion 30 may include a plurality of sutures 32 (FIG. 3B). Sutures 32 may grasp onto sclera 2 when expandable portion 30 expands in a vertical and/or a horizontal direction.

Components of instrument 10 may be made of any suitable metal, polymer, and/or combination of metals and/or polymers. Exemplary metallic materials may include stainless steel, nitinol, titanium, and/or alloys of these metals. Exemplary polymeric materials may include polyetheretherketone (PEEK), polyimide, and polyethersulfone (PES). In some examples, tubular shaft 14 may be made of a rigid material, semi-rigid material, or flexible material, wherein such material may be expandable and/or may allow for various configurations as discussed herein. Materials of instrument 10 may be any biocompatible material that may be sterilized.

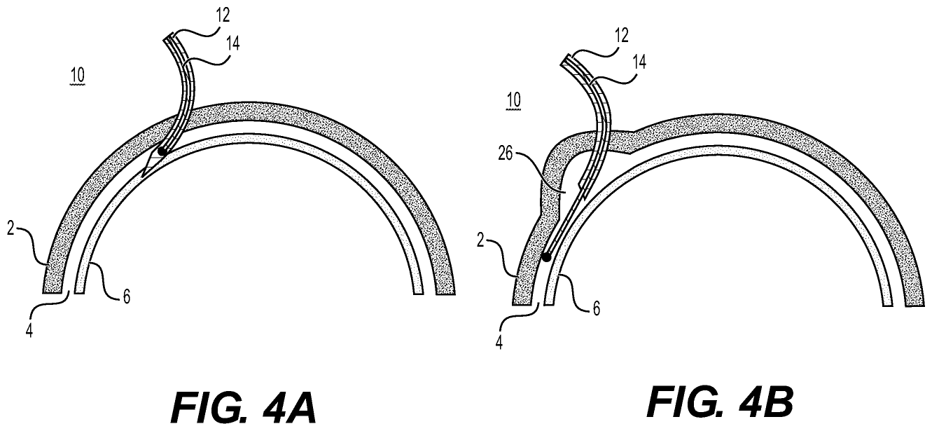

Referring to FIGS. 4A and 4B, instrument 10 may have a curved or fishhook shape. The curvature of instrument 10 may allow for insertion of instrument 10 at a desired depth between sclera 2 and choroid 6, such that the user may rotate and/or lift instrument 10 to pull a portion of sclera 2 away from SCS 4, creating expanded portion 26. As shown in FIG. 4B, SCS 4 may expand outwards, i.e., away from choroid 6, instead of inwards, which may help avoid adverse effects when injecting a drug into the eye tissue. Tubular shaft 14 may be advanced to further separate sclera 2 and choroid 6 (FIG. 4B), which may enable the injected drug to more evenly spread between sclera 2 and choroid 6. A bevel or opening of needle 12 may be positioned in any direction relative to the curvature of instrument 10, including toward the center of curvature, away from the center of curvature, or in any direction therebetween. Further, instrument 10 may include a magnetic element or may be formed in whole or in part from a magnetic material. When inserted into SCS 4 and while positioned between sclera 2 and choroid 6, a magnet external to the patient's eye may be used to guide instrument 10 by acting on the magnetic element or magnetic material. For example, the external magnet may be positioned outside of the patient's eye near an outside surface of sclera 2. The external magnet may exert a magnetic force on needle 12, thereby urging needle 12 toward sclera 2 and maintaining the portion of needle 12 located within SCS 4 in an orientation that is parallel or nearly parallel with sclera 2 to avoid inadvertently penetrating choroid 6.

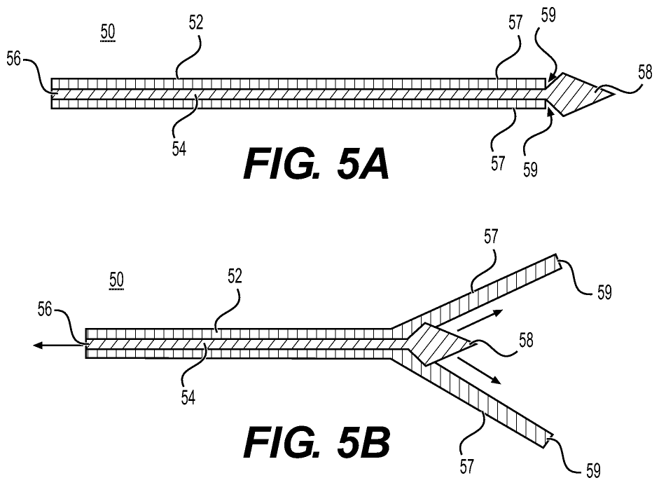

Various configurations of instrument 10, as well as the components of instrument 10 are described herein. FIGS. 5A and 5B represent an alternative embodiment, wherein instrument 50 may include a needle 52 and a shaft 54 (e.g., a tubular shaft). Needle 52 may include a passage 56 and a distal end 57 and tubular shaft 54 may include a distalmost tip 58. Distalmost tip 58 may be a sharp or piercing tip configured to penetrate a tissue layer of the eye. Distal end 57 may have a substantially atraumatic or blunt tip 59. As shown in FIGS. 5A and 5B, distalmost tip 58 may include a plurality of angled/curved surfaces such that once needle 52 is inserted into the sclera, tubular shaft 54 may be pulled backwards through passage 56. While tubular shaft 54 is pulled through passage 56, the angled/curved surfaces of distalmost tip 58 may force a portion of distal end 57 to widen (FIG. 5B), which in turn, may separate the sclera and choroid layers, allowing a drug to flow more readily from the injection site. Tubular shaft 54 may also be pushed back through 56 to its original orientation, as shown in FIG. 5A. The drug may be delivered from one or more openings in distal tip 58, as depicted by the arrows in FIG. 5B. Instrument 50 may include a locking/unlocking mechanism to prevent tubular shaft 54 from being pushed backwards through passage 56 while instrument 50 is inserted into the sclera. In other examples, portions of needle 52 may be shaped or curved to resemble a shape or curvature of the eye.

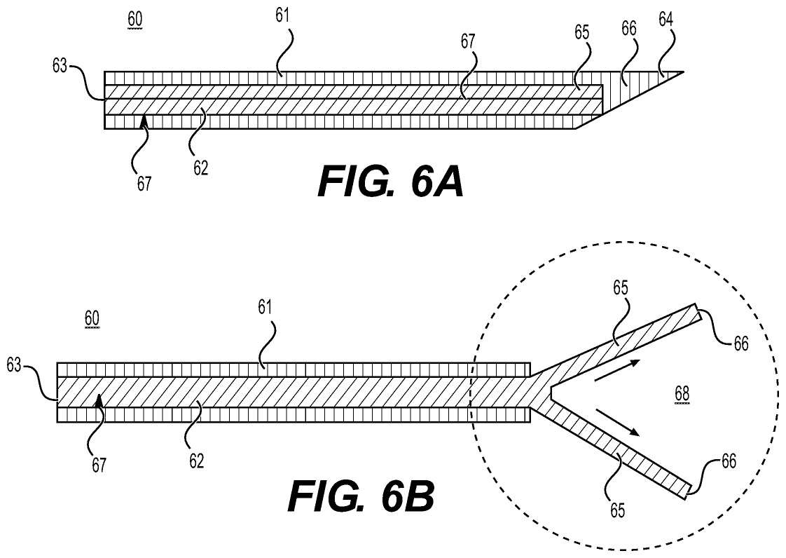

FIGS. 6A and 6B represent an alternative embodiment, wherein instrument 60 may include a needle 61 and a tubular shaft 62. Needle 61 may include a passage 63 and a distalmost tip 64 and tubular shaft 62 may include a distal end 65. Distalmost tip 64 may be a sharp tip or needle configured to penetrate a tissue layer of the eye. Distal end 65 may have a substantially atraumatic or blunt tip 66. As shown in FIG. 6A, tubular shaft 62 may be configured such that a wedge 67 of instrument 60 may engage with tubular 62 once instrument 60 is inserted into the sclera of the eye, wedge 67 forcing a portion of tubular shaft 62 to spread (FIG. 6B) within the plane of the SCS to separate the sclera and choroid layers, and allow for a drug to flow and spread more readily form the injection site. For example, tubular shaft 62 may include a split 68 allowing a portion of tubular shaft to expand, as shown in FIG. 6B. FIG. 6B is a top view of instrument 60 showing the expanded distal end 65 of tubular shaft 62. Wedge 67 may be any appropriate shape configured to engage with and force apart a portion of tubular shaft 62. In some embodiments, wedge 67 may be omitted, and distal end 65 may be configured to be self-expanding to form split 68 once distal end 65 is advanced out of passage 63.

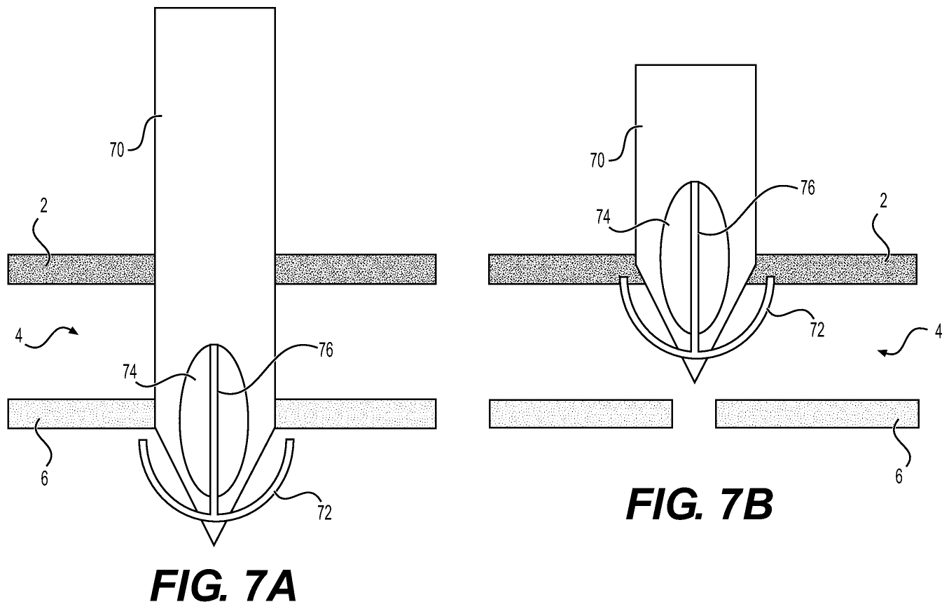

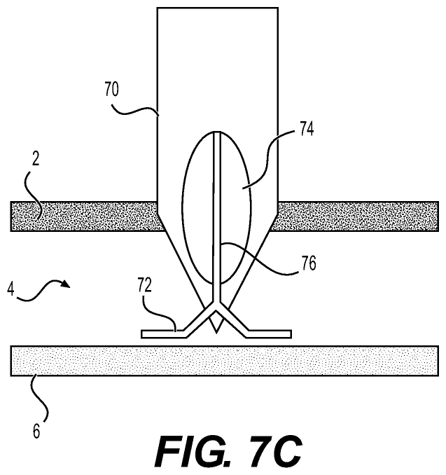

FIGS. 7A-7C show exemplary components for a distal end of tubular shaft 76 extending out from needle 70. Needle 70 may include at least one opening 74. A distal end of tubular shaft 76 may include an anchor 72. Anchor 72 may have a concave curvature. The user may insert needle 70 into sclera 2 and choroid 6 (FIG. 7A) and then pull needle 70 back towards the user, away from choroid 6 of the eye. Upon retraction of needle 70, anchor 72 activates and engages with an inner surface of sclera 2 (FIG. 7B), which may stop needle 70 at a desired depth within SCS 4. Anchor 72 is able to stop once it hits sclera 2 because of the different mechanical properties between sclera 2 and other, softer layers, of the eye tissue. Anchor 72 may also pull sclera 2 away from choroid 6, thereby increasing a portion of SCS 4. In some embodiments, anchor 72 may be formed in whole or in part of a magnetic material, such that a magnet external to the patient's eye may be used to pull sclera 2 away from choroid 6. While this embodiment may require penetration of choroid 6, it may allow the user to easily reach a correct depth of insertion without the use of precise device geometries. In another embodiment, as shown in FIG. 7C, anchor 72 may be configured to prevent penetration of choroid 6. For example, anchor 72 may have a planar or otherwise atraumatic shape or configuration to abut against choroid 6 without penetrating the tissue layer. Anchor 72 may be pushed out of needle 70 manually or anchor 72 may automatically extend from needle 70 upon a decrease in penetration force. In some examples, anchor 72 may be formed of a flexible material or a semi-flexible material, such that portions of anchor 72 may bend. For example, portions of anchor 72 may bend towards choroid 6 as needle 70 is retracted/removed from the tissue layers once injection is complete.

In some embodiments, anchor 72 may be configured to engage with an inner surface of choroid 6. In such embodiments, the user may insert needle 70 into sclera 2 and choroid 6 (FIG. 7A) and then pull needle 70 back towards the user. Upon retraction of needle 70, anchor 72 may activate and engage with an inner surface of choroid 6, thereby separating choroid 6 from a retina underneath. As choroid 6 is formed of soft tissue, anchor 72 may be configured to separate choroid 6 from the retina without tearing or rupturing choroid 6. Such a configuration may be particularly useful for performing treatments such as gene therapy in a subretinal space to address various conditions such as retina detachments and the like.

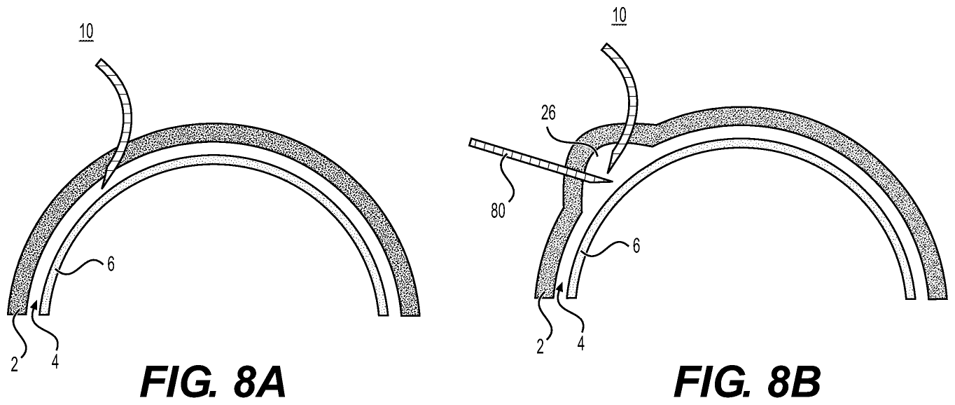

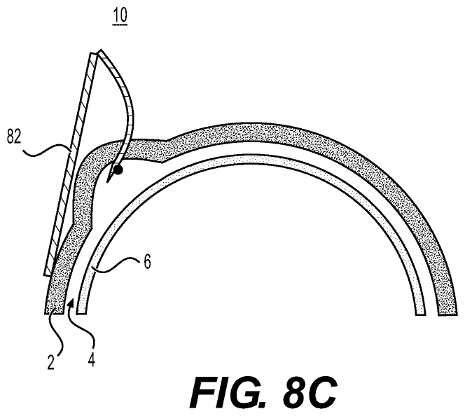

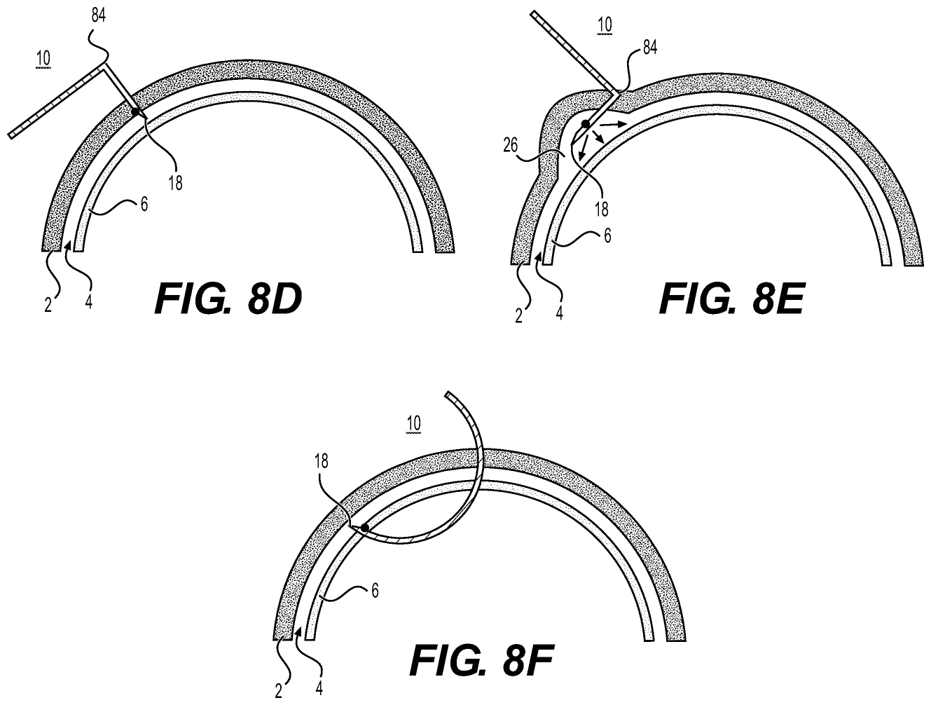

FIGS. 8A-8F show exemplary embodiments of instrument 10 and methods of delivering a medicament to SCS 4 of the eye. Instrument 10 may include all or some of the features as discussed above. As shown in FIG. 8A, instrument 10 may have a curved or fishhook shape. The curvature of instrument 10 may allow for insertion of instrument 10 at a desired depth between sclera 2 and choroid 6, such that the user may rotate and/or lift instrument 10 to pull a portion of sclera 2 away from SCS 4, creating expanded portion 26. As shown in FIG. 8B, SCS 4 may expand outwards, i.e., away from choroid 6, instead of inwards, which may help avoid adverse effects when injecting a drug into the tissue. In some examples, a needle 80 may be inserted into expanded portion 26. The use of instrument 10 in conjunction with needle 80 may require less user precision because of the increased distance, i.e., expanded portion 26, between sclera 2 and choroid 6. However, use of instrument 10 in conjunction with needle 80 may need to be precisely controlled since this method utilizes two insertion sites, one for instrument 10 and a second for needle 80.

FIG. 8C shows an alternative second instrument, stabilization leg 82, for use in conjunction with instrument 10. A user may attach stabilization leg 82 to instrument 10, such that the use of both components may control how deep instrument 10 is inserted through sclera 2 and SCS 4. Compared to the systems and methods shown in FIG. 8B, the use of stabilization leg 82 utilizes one insertion site, which may reduce risks of infection, discomfort, and/or trauma in a patient. Stabilization leg 82 further provides additional user control.

FIGS. 8D and 8E show an alternative embodiment of instrument 10 including a bend 84 positioned proximally of distalmost tip 18. Bend 84 may control how deep instrument 10 may be inserted through sclera 2 and SCS 4. Once instrument 10 is inserted, a user may rotate instrument 10 to simultaneously pull a portion of sclera 2 away from choroid 6, while injecting a volume of a drug in to expanded portion 26. FIG. 8F shows an alternative embodiment of instrument 10 configured in a substantially U-shape. The U-shape of instrument 10 may control how deep instrument 10 may be inserted through sclera 2, SCS 4, and choroid 6. Once instrument 10 is inserted, a user may push/rotate instrument 10 causing distalmost tip 18 to point upwards towards sclera 2, without making a second insertion into sclera 2. Differences in mechanical properties between the sclera and other softer layers may give the user tactile feedback that indicates the user may need to stop needle advancement before it penetrates the sclera layer again from the inside. While the U-shape may enable the user to find a correct injection depth without the need for ultra-precise device geometries, utilizing the U-shape instrument penetrates choroid 6 in some embodiments.

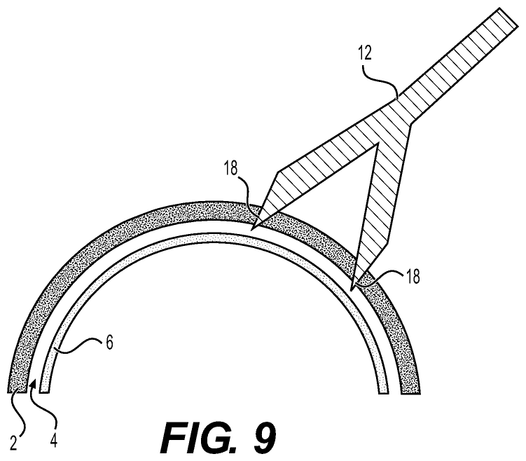

In some examples, needle 12 may include more than one distalmost tip 18. Referring to FIG. 9, distalmost tip(s) 18 of needle 12 may form a Y-shape, which may allow needle 12 to have two injection sets into sclera 2. Distalmost tip(s) 18 may straddle an area of sclera 2, allowing the drug to spread to a larger area of SCS 4. Openings in distalmost tip(s) 18 may be oriented in various directions, including outwardly from a main shaft of needle 12, inwardly toward the main shaft, or in various directions there between. The openings may be oriented to maximize flow of a drug from needle 12 and the surface area over which the drug flows. Additionally, though distalmost tip(s) 18 are shown in FIG. 9 as extending straight from needle 12, distalmost tip(s) 18 may collectively form a semi-circular shape resembling the shape of the outer surface of the patient's eye. Such a configuration may allow insertion of distalmost tip(s) 18 near a limbus of the eye and upon injection of the drug through needle 12, the drug to flow toward the back of the eye.

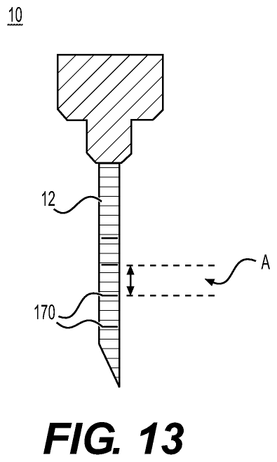

When using an apparatus or system as disclosed herein, differences in mechanical and/or chemical properties between the vitreous, choroid, and sclera may enable the user to feel tactile feedback when the apparatus, system, or components thereof reach the internal surface of the sclera. In other words, based on the properties of the tissue layers, the user may know once the apparatus, system, or components thereof, are inserted to the correct depth. For example, the sclera may be about 10 times stiffer than the choroid. While a standard needle may be inserted at an angle into the eye, following a curvature of the eye, and the user may use tactile feedback as described above, such a method may increase the risk of trauma to the eye. Referring to FIG. 13, needle 12 may include geometric features 170 configured to provide tactile feedback to the user. Geometric features 170 may be notches or ribs and may be placed at set intervals, for example, every 100 μm (represented by A in FIG. 13). In addition to providing tactile feedback to the user, geometric features 170 may further provide audible feedback to the user. For example, geometric features 170 may produce clicking sounds upon insertion to one or more depths into the eye or into one or more spaces in the eye. In some embodiments, geometric features 170 may include one or more sensors configured to detect a depth of insertion of needle 12. Based on signals generated by the one or more sensors indicative of insertion to one or more depths into the eye, audio may be generated to alert the user of a depth of insertion. The audio may be generated by any audio generating device, such as a small electronic speaker or the like, located on needle 12, on a needle hub, on a housing of a syringe, or positioned nearby and separate from needle 12. Geometric features 170 may therefore allow a user to use needle 12 for injections into any of several spaces of the eye, such as the suprachoroidal space, vitreous humor, or other spaces.

The differences in properties of the tissue layers may also assist in controlling drug flow. For example, needle 12 may have a plurality of openings 150 as shown in FIGS. 11A and 11B. In some examples, openings 150 may be configured around a circumference and/or down a length of needle 12. Once needle 12 may be inserted into the tissue layers, the different chemical and/or mechanical properties of the tissue layers may block openings 150 and prevent a volume of the drug from flowing out of needle 12 and into areas of the tissue layers other than the SCS.

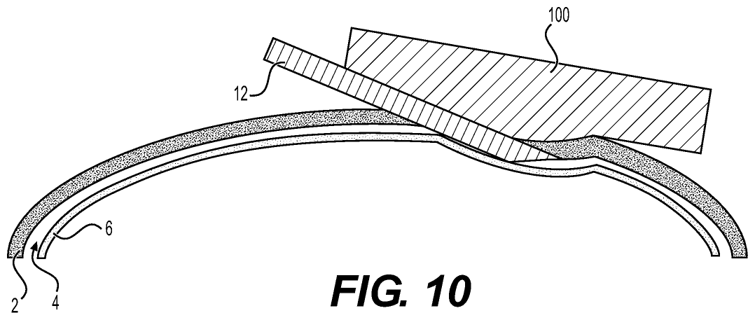

Referring to FIG. 10, an external portion 100 may be utilized along with needle 12. External portion 100 may comprise a planar surface having a projection for deforming one or more of sclera 2 or choroid 4. For example, the projection may be a rounded surface. External portion 100 may be applied to the outer surface of the eye to create a deformation in one or more of sclera 2 or choroid 4. As shown in FIG. 10, once external portion 100 is applied to the outer surface of the eye, needle 12 may be inserted at an appropriate angle and/or distance from the deformed area of sclera 2 and/or choroid 4, such that needle 12 may become wedged between sclera 2 and choroid 4. While the use of external portion 100 may assist with inserting needle 12 at a correct depth, external portion 100 does not necessarily increase an area of SCS 4 for drug delivery.

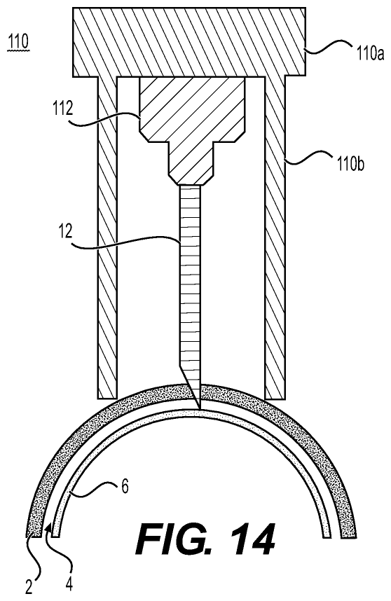

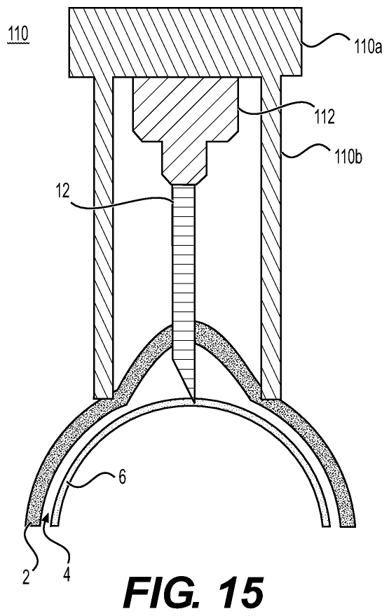

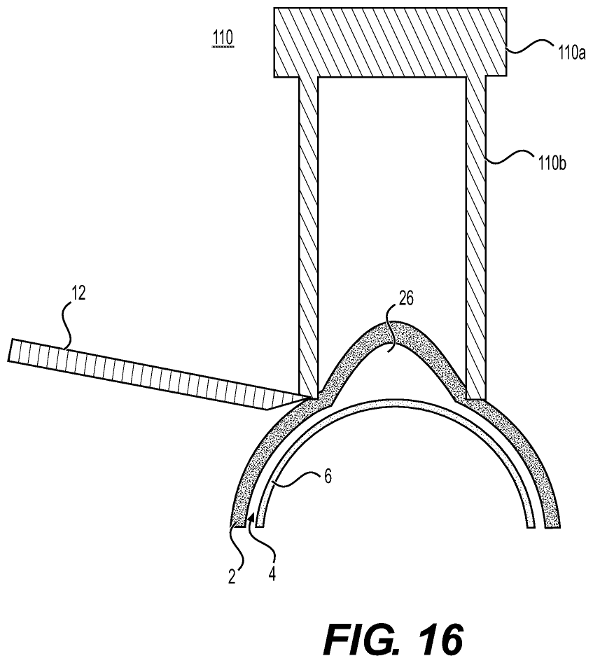

FIGS. 14-17 show exemplary embodiments of the present disclosure utilizing a chamber 110 along with needle 12. Chamber 110 may be configured to draw, grab, or pinch a portion of sclera 2. For example, chamber 110 may have a circular shape and/or components that may grab a portion of sclera 2 and draw such portion of sclera 2 therein chamber 110. Referring to FIG. 14, chamber 110 may include a portion 110a and a shield 110b extending from a distal surface of portion 110a. Portion 110a may be a fixed table, tray, or forehead rest. Needle 12 may be connected to chamber 110 via any appropriate means, for example, a needle hub 112. Chamber 110 may be activated automatically or manually by the user such that chamber 110 may pinch a portion of sclera 2 and pull it into chamber 110, i.e., away from choroid 6 (as shown in FIG. 15). In some examples, chamber 110 may be configured to apply a suction to sclera 2. For example, chamber 110 may include a vacuum, such as a dynamic vacuum pull or a static vacuum pull. Referring to FIG. 15, chamber 110 may include a vacuum to apply a suction to a portion to sclera 2. Once a portion of sclera 2 is pulled therein chamber 110 and away from choroid 6, needle 12 may be inserted into such portion of sclera 2. FIG. 16 shows another embodiment where chamber 110 may be applied to a portion of sclera 2. Chamber 110 may include a vacuum to pull a portion of sclera 2 upwards into chamber 110. Needle 12 may be inserted at a base of chamber 110, for example, at a position distally of shield 110b.

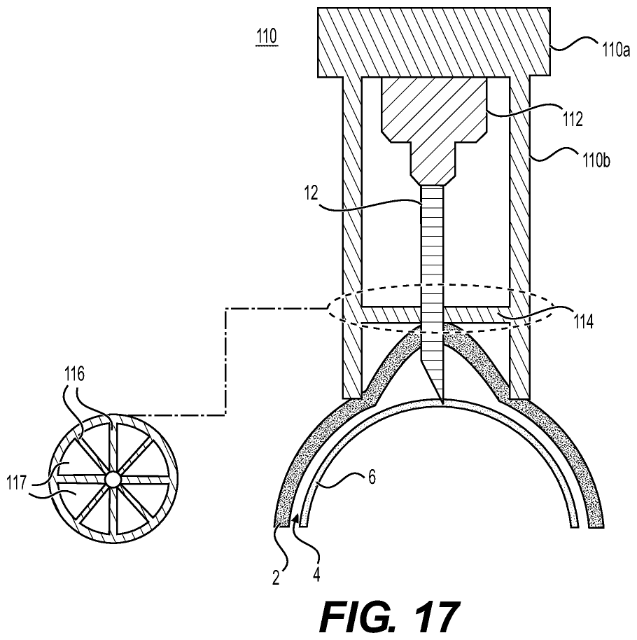

In another embodiment, chamber 110 may include a stop component 114 configured to limit proximal advancement of sclera 2 into chamber 110 (FIG. 17). For example stop component 114 may surround a portion of needle 12. Stop component 114 may include a plurality of extensions 116 extending from a sidewall of chamber 110 towards a center of chamber 110. Stop component 114 may also include a plurality of openings 117 configured to facilitate application of a suction, e.g., from a vacuum, to sclera 2. Stop component 114 may further be configured to allow a user to adjust an angle of the injection. For example, upon turning stop component 114 and/or chamber 110 about a longitudinal axis of needle 12, the angle of penetration of sclera 2 by needle 12 may be adjusted. In some embodiments, a user may adjust the angle of penetration from about 90° relative to the surface of sclera 2 (as shown in FIG. 17) to about 45° by rotating stop component 114 and/or chamber 110. Stop component 114 may cause adjustments to the angle of penetration, for example, by deflecting needle 12 when needle 12 has a flexible construction or is formed of a flexible material.

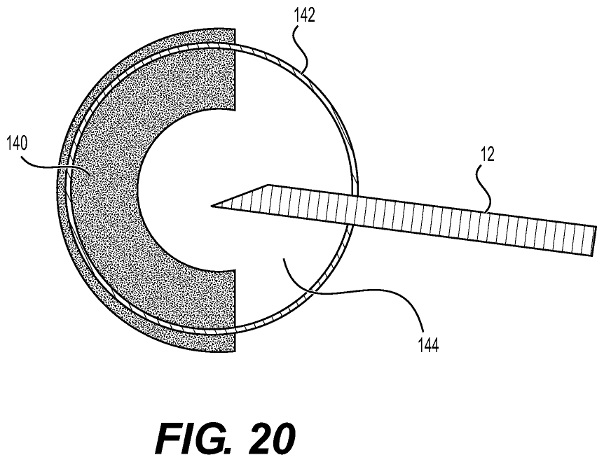

As described above, shield 110b of chamber 110 may include a circular cross-section configuration. Referring to FIG. 20, instead of applying a suction to the entire circular section of sclera 142 enclosed by shield 110b, the suction may be applied in a horseshoe or half-circle shape 140. In such case, shield 110b of chamber 110 may include a semi-circular cross-sectional configuration. Applying the suction in a horseshoe shape 140 may raise a portion of sclera 142 upwards into section 144. The user may then insert needle 12 horizontally (into sclera 142 and tangent to the natural eye surface) into a center and between the two sides of horseshoe shape 140. Further insertion of needle 12 may wedge needle 12 between the sclera and choroid layers. The systems and methods of use disclosed herein, for example at FIGS. 14-17 and 20, may be minimally invasive to the patient, while creating additional space within SCS 4 without disturbing choroid 6. The precision of the needle insertion may be less important because the area of SCS 4 has been increased via use of the vacuum.

Systems and methods as described herein, which may comprise chamber 110, may be utilized to prevent a drug from being pulled out of the needle or apparatus. For example, referring to FIG. 15, needle 12 may be partially inserted into a portion of the eye. Chamber 110 may suction a portion of sclera 2 and may pull such portion of sclera 2 up to cover distalmost tip 18. In another example, a delivery system and/or apparatus according to the present disclosure may have a friction force greater than chamber 110, such that chamber 110 may pull up a portion of sclera 2 while allowing a drug to flow out of needle 12 and into SCS 4. In other examples, a valve, e.g., a shut off valve, may be present on needle 12, needle hub 112, or any component of a device or apparatus according to the present disclosure. The valve may be used to block and unblock the flow of drug from needle 12. For example, after chamber 110 is applied to sclera 2 and a portion of sclera 2 may be suctioned and pulled up into chamber 110, needle 12 may be inserted and that a valve may be utilized to unblock the flow of a drug from needle 12 into SCS 4 and then blocked to halt the flow of a drug from needle 12.

In some embodiments, chamber 110 may rest against the outer surface of the eye to act as a guide for the user (FIG. 19). User may rest shield 110b of chamber 110 against the outer surface of the eye and then advance needle 12 out of needle hub 112 and into sclera 2. As shown in FIG. 19, chamber 110 may include a dial or crank 220 for advancing needle 12. The apparatus and method disclosed herein, for example at FIG. 19, may closely control the needle insertion depth.

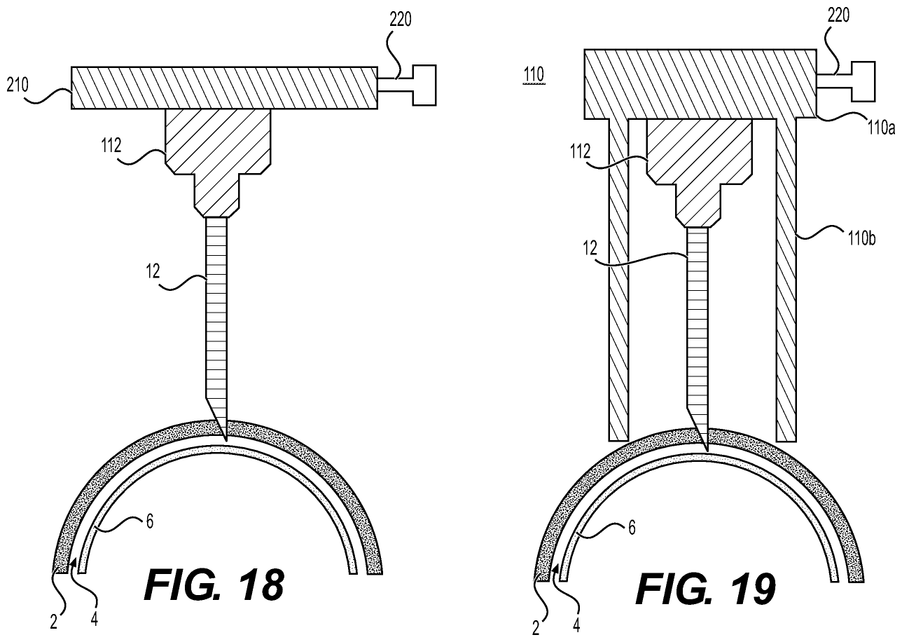

In another example, as shown in FIG. 18, needle hub 112 may house a portion of needle 12, wherein needle hub 112 may be attached to a holder 210. Holder 210 may be attached to a table, tray, or forehead rest, which may be fixed to a stable, external surface, to aid the user. Holder 210 may include a dial or crank 220 for advancing needle 12. Needle hub 112 may include a feedback mechanism, for example a force sensor, for stopping needle 12 from extending from holder 210 when it senses a decrease in penetration resistance. As discussed above, penetration resistance may refer to the mechanical and/or chemical properties of the ocular tissue layers. Dial 220 may be used to insert needle 12 into the eye in a controlled manner, until the feedback mechanism notifies the user to stop due to the decrease in force and/or penetration resistance. The feedback mechanism may be a feedback loop so that insertion of needle 12 may automatically stop.

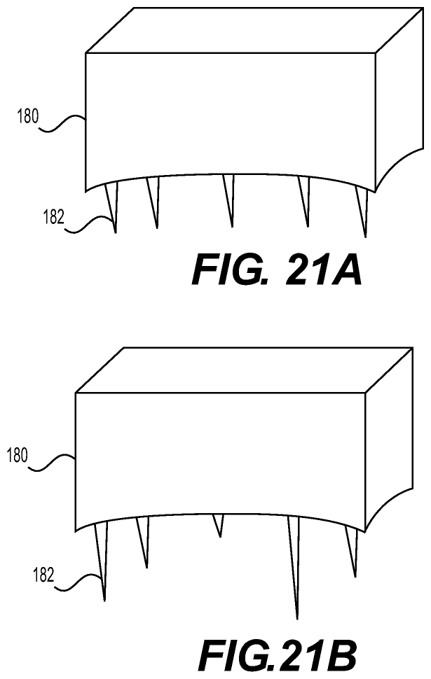

Referring to FIGS. 21A and 21B, systems and methods described herein may include a microneedle hub 180. Microneedle hub 180 may be substantially rectangular and include a plurality of curved surfaces. Microneedle hub 180 may include a plurality of microneedles 182. As shown in FIGS. 21A and 21B, microneedles 182 may be of varying lengths and thicknesses. In another example, microneedles 182 may be of varying angles, wherein microneedles 182 may be of varying angles relative to a surface of microneedle hub 180. In some embodiments, microneedles 182 may be angled about 45° relative to microneedle hub 180. In some embodiments, each of microneedles 182 may be angled in the same direction. In some embodiments, one or more of microneedles 182 may be angled in different directions. Microneedle hub 180 may provide a staged firing of different sets of microneedles, similar to a tattoo needle. This stage firing may spread a drug over a large area of the ocular tissue. Varying lengths of microneedles 182 (FIG. 21B) may provide the user with flexibility when dealing with varying thicknesses of the ocular tissue layers, e.g., the sclera. In some embodiments, microneedles 182 may include an opening (not shown) at the distal end of each of microneedles 182. The openings of microneedles 182 may be oriented in varying directions such that a drug may flow in multiple directions relative to microneedle hub 180. In some embodiments, microneedles 182 may be formed of a lyophilized drug such that upon insertion into a patient, microneedles 182 may dissolve, thereby releasing the drug. Microneedle hub 180 may be utilized alone or in conjunction with any of the devices, systems, and methods disclosed herein.

In some embodiments, microneedle hub 180 and/or microneedles 182 may include a spring-loaded mechanism. When microneedles 182 are pressed against an eye, the spring-loaded mechanism may cause microneedles 182 to deflect angularly relative to microneedle hub 180. In some embodiments, microneedles 182 may be arranged in a circular or semicircular formation and may deflect in an outward direction relative to the formation. Deflection of microneedles 182 may allow microneedles 182 to penetrate the eye at sufficiently shallow angles, separate the sclera 2 and choroid 6 layers to allow for better and increased drug flow, and may further maximize an area over which a drug is distributed.

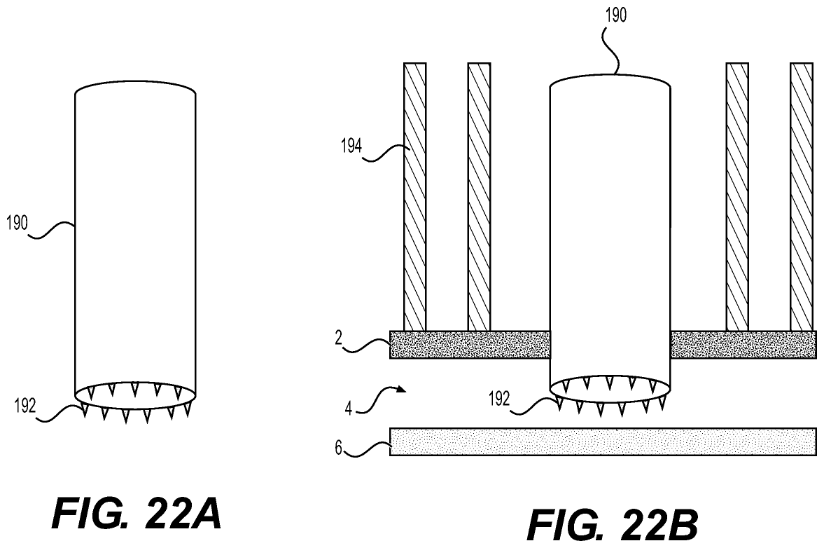

Another embodiment as shown in FIGS. 22A and 22B, include a needle tubing 190 with a needle tip 192. Needle tubing 190 may have a cylindrical shape and needle tip 192 may be a serrated needle tip. Needle tip 192 oscillates on the outer surface of the eye, which may allow needle tip 192 to cut through sclera 2 without penetrating choroid 6. In some examples, needle tubing 190 with needle tip 192 may be surrounded by a chamber 194 as described above, where chamber 194 may include a vacuum. Chamber 194 may be applied in a circular shape around needle tubing 190 to pinch onto and grab sclera 2 and lift a portion of sclera 2 upwards. The vacuum of chamber 194 may create a seal on the external surface of sclera 2, which may allow chamber 194 to grab onto a portion of sclera 2.

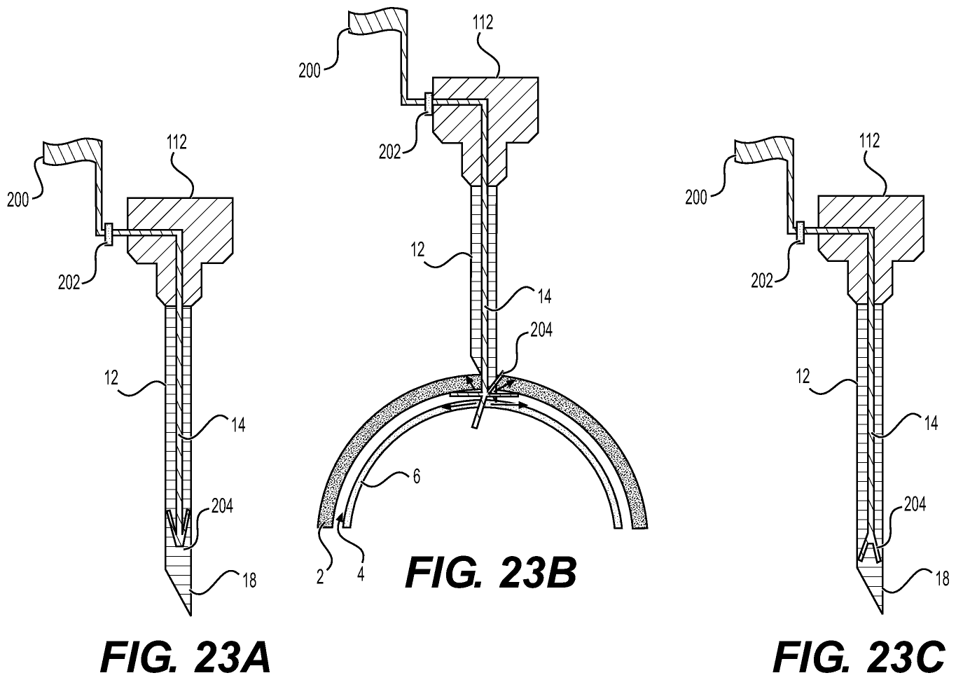

FIGS. 23A-23C represent another embodiment wherein needle 12 may be connected to needle hub 112. Tubular shaft 14 may include a lever 200 and a stop 202. Lever 200 and stop 202 may be positioned at a proximal end of tubular shaft 14. A distal end of tubular shaft 14 may include a pair of arms 204. Distalmost tip 18 of needle 12 may be sharp to penetrate sclera 2. FIG. 23A shows the device prior to injection. During injection (FIG. 23B) lever 200 may be activated to push tubular shaft 14 through needle 12 and into SCS 4. Lever 200 may push tubular shaft until stop 202 is flush with needle hub 112 to seal the device during delivery. Once inserted, arms 204 may mechanically separate sclera 2 and choroid 6, and in turn guide the drug outwards into SCS 4 from the injection site. When injection is complete (FIG. 23C), lever 200 may be pulled upwards to remove arms 204 from the ocular tissue layers. In some examples, arms 204 may be formed of a thread-like hydrogel and may be configured to detach from tubular shaft 14 when positioned in SCS 4. Arms 204 may be loaded with a drug that is released upon hydration. Arms 204 may be hydrated by injection of a hydrating solution into a proximity of arms 204 or may be hydrated naturally over time due to exposure to naturally occurring fluid in the eye.

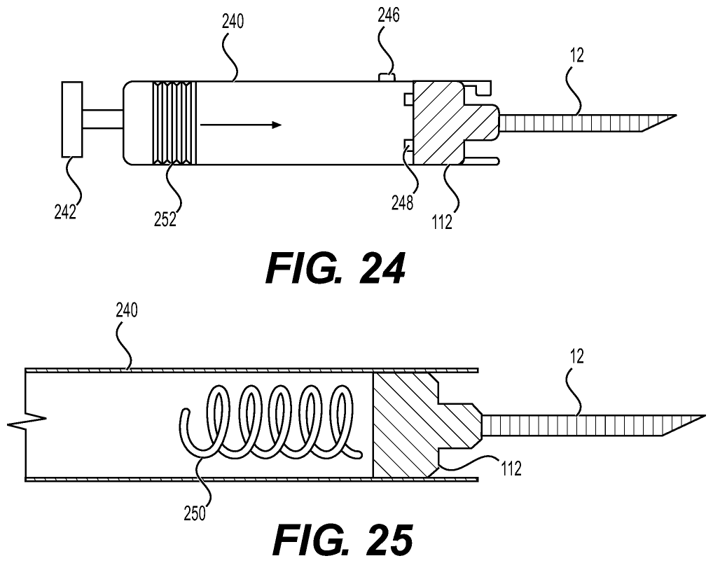

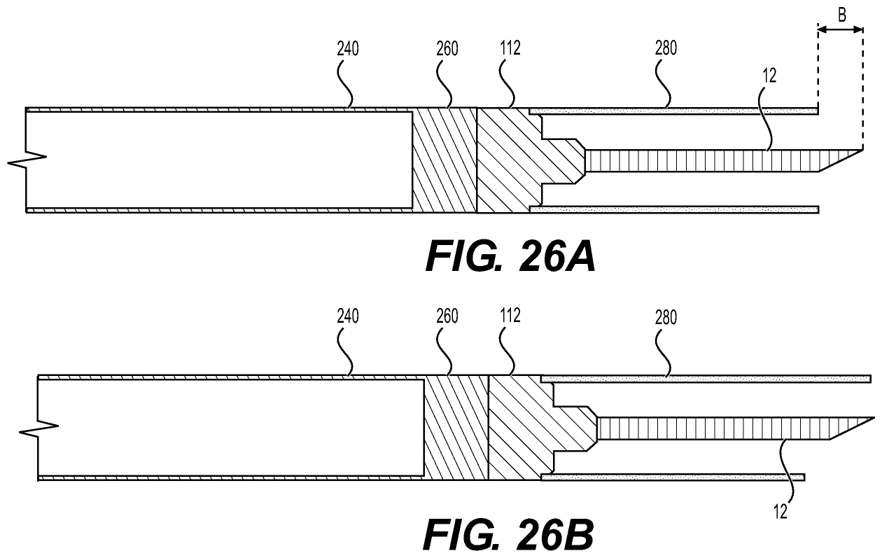

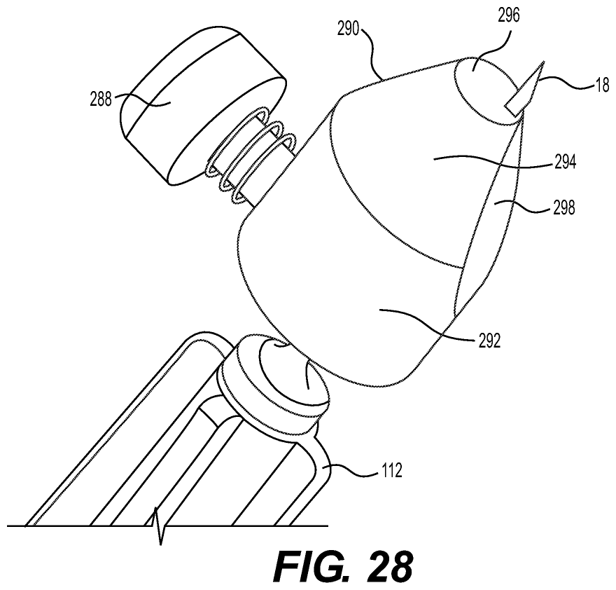

FIGS. 24-27 represent embodiments of the present disclosure wherein needle 12 may be connected to needle hub 112. The device includes a housing 240. Housing 240 may have a cylindrical shape and may contain needle hub 112, and any additional components, e.g., a syringe, connector 260 (e.g., a luer lock adaptor on a syringe) or spring 250 that may be connected to needle hub 112. Referring to FIG. 24, housing 240 may contain a piston 252, plunger rod 242 on a proximal end of housing 240, an indicator 246, and feedback mechanism 248. Spring 250 may be in line with needle 12, such that spring 250 provides resistance as the user is inserting needle 12 through the sclera. Once needle 12 is inserted through the sclera, the spring force drops and may provide a signal to the user indicating that the user should stop inserting needle 12. The signal may be indicator 246 or any appropriate signal. For example, indicator 246 may be a LED light or a noise. Feedback mechanism 248 may be a stop or a brake that prevents spring 250 from pushing needle hub 112 past a pre-set limit in housing 240. In some examples, needle hub 112 may be configured to not move towards a distal end of housing 240 and only move back towards a proximal end of housing 240. The device may also include an external shaft 280 (FIGS. 26A and 26B) that may snap onto needle hub 112. Alternatively, external shaft 280 may attach directly to needle 12. External shaft 280 may have a cylindrical shape enclosing at least a portion of needle 12. In some embodiments, external shaft 280 may be formed of a transparent material, allowing the user to see needle 12 through external shaft 280. This may allow the user to more easily observe a positioning of needle 12 during an injection. The distance between a distal end of external shaft 280 and the distalmost tip of needle 12, represented by B in FIG. 26A, may be used to control the depth of needle insertion. Distance B may be adjustable. For example, external shaft 280 may have a threaded component that defines Distance B, external shaft 280 may include a mechanism that makes Distance B adjustable, external shaft 280 may have markings, e.g., color codes, that indicate the length of Distance B to the user; or external shaft 280 may be a disposable component. In some examples, a number of external shafts 280 may be provided to a user, wherein each of external shafts 280 may provide a different Distance B, such that the user may choose the correct shaft component based on an estimation of the patient's sclera thickness. In another example, external shaft 280 may have an angled edge (FIG. 26B), to enable needle insertion at a desired angle. In another example, the device may operate automatically such that needle 12 is inserted into the eye until feedback mechanism 248 (as shown in FIG. 24) stops needle 12 from being inserted further. Such automatic operation may improve control of needle 12 and may further control an overall injection rate. In still another example, external shaft 280 may be shaped similarly to adaptor 290 (shown in FIGS. 28-30 and described in further detail hereinafter), such that it includes a surface that may be placed against and mate with a sclera of a patient's eye. In still another example, external shaft 280 may translate relative to needle hub 112 and needle 12 and may provide an audible feedback to a user. For example, translation of external shaft 280 may produce a clicking sound audible to the user when the translation reaches a predetermined limit. The predetermined limit may be, for example, a point at which needle hub 112 nears the distal end of external shaft 280. In still another example, the embodiments shown in FIGS. 24-27 may be combined with any of the feedback features described herein previously which provide the user with indications of whether needle 12 has been inserted into the target location.

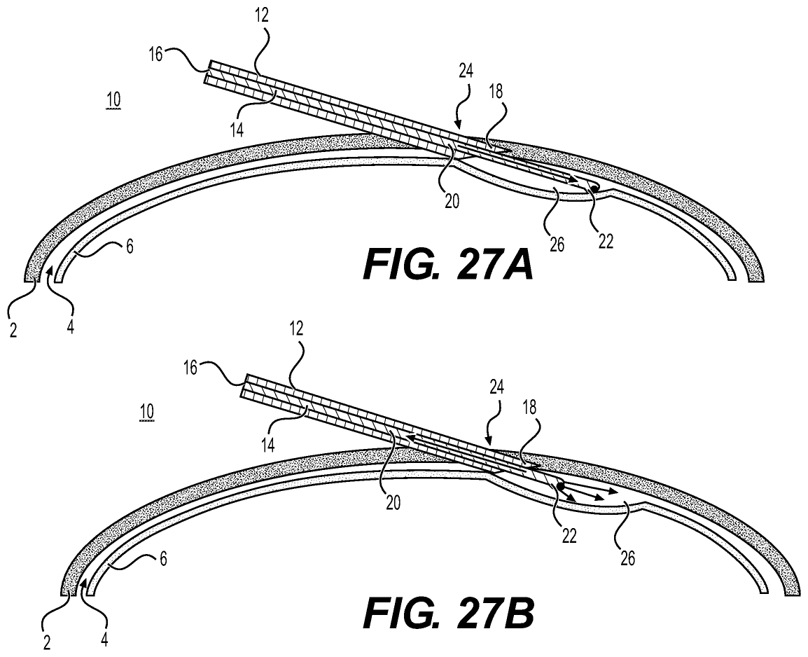

Various methods of delivering a drug to a SCS of a patient's eye are disclosed throughout the discussion of the devices and systems herein. An example of delivering a medicament to a suprachoroidal space of a patient's eye using instrument 10 is shown in FIGS. 27A and 27B. Instrument 10 may be inserted through injection site 24, such that distalmost tip 18 of needle 12 may cut a portion of sclera 2. Once instrument 10 is in SCS 4, tubular shaft 14 may be inserted into SCS 4 until it reaches a certain distance from insertion site 24 and expands a portion of sclera 2 and/or choroid 6, thereby increasing an area of SCS 4 (FIG. 27A). Once tubular shaft 14 is inserted into SCS 4, a volume of drug may be injection into the area of SCS 4. Tubular shaft 14 may be retractable, as shown in FIG. 27B. As tubular shaft 14 is retracted, a volume of a drug may be simultaneously injected from tubular shaft 14 into the area of SCS 4, such that the drug may be the space left by tubular shaft 14. Instrument 10, needle 12, and tubular shaft 14 may include any of the components as discussed above.

Methods disclosed herein may be pressure-controlled. For example, an injection or infusion rate may be based on a pressure feedback. Pressure in the SCS may be limited to prevent increased levels of pressure that may cause damage to the ocular tissues of the eye. A pressure-controlled injection may also allow for a longer duration of drug delivery away from the injection site within the SCS. In other examples, the apparatus and/or devices discussed herein may be connected to a pump/electromechanical device that may monitor the pressure in the entire system. A pressure-controlled injection may also control a flow rate of a drug into the SCS such that the pressure of the flow rate may not exceed a certain pressure, e.g., IOL pressure.

Referring to FIGS. 28-30, systems and methods described herein may include an adaptor 290. Adaptor 290 may be a component configured to surround a shaft of needle 12. Adaptor 290 may be positioned toward distalmost tip 18 relative to needle hub 112 to which needle 12 may be connected. In another configuration, needle 12 is connected to a container (not shown) for a medicament, for example, a syringe. In some examples, needle 12 may be a staked needle. In other examples, needle hub 112 may be disposed between the container (not shown) and needle 12. Adaptor 290 may include an intermediate surface 292 which defines a substantially cylindrical portion of adaptor 290. When adaptor 290 is positioned to surround a portion of needle 12, a longitudinal axis of the substantially cylindrical portion of adaptor 290 may extend parallel to a longitudinal axis of needle 12. For purposes of this disclosure, the longitudinal axis of the substantially cylindrical portion should be understood to be a longitudinal axis of adaptor 290.

Adjacent to intermediate surface 292, adaptor 290 may include an angled distal surface 294 disposed toward a distal end of adaptor 290 relative to intermediate surface 292. Angled distal surface 294 may define a substantially frustoconical portion or a partially frustoconical portion of adaptor 290. Angled distal surface 294 may be oriented at an angle ranging from about 30 degrees to about 60 degrees relative to the longitudinal axis of adaptor 290, at an angle ranging from about 40 degrees to about 50 degrees relative to the longitudinal axis of adaptor 290, or at about a 45 degree angle relative to the longitudinal axis of adaptor 290, for example.

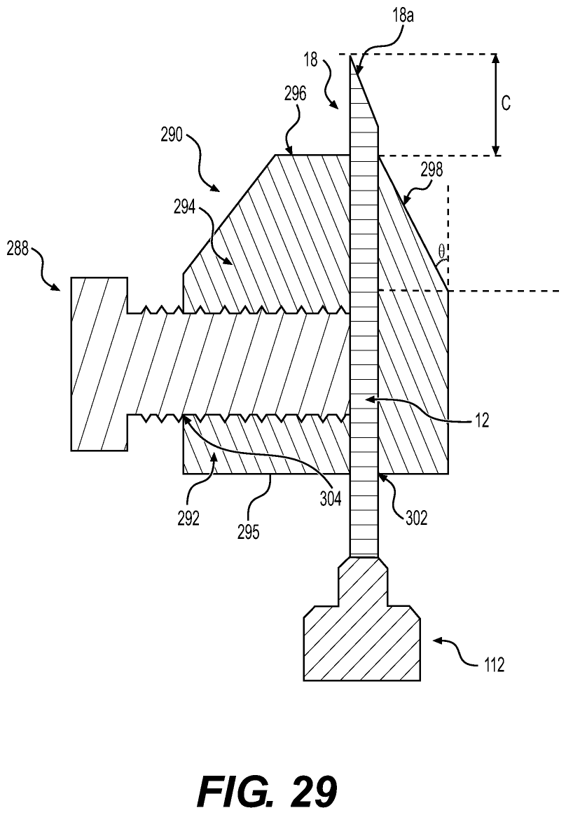

Adaptor 290 may further include an outermost slanted surface 298. Outermost slanted surface 298 may be a planar surface adjacent to intermediate surface 292 and/or angled distal surface 294. Alternatively, outermost slanted surface 298 may be a convex surface configured to be placed against and mate with a sclera of a patient's eye. As shown in FIG. 29, outermost slanted surface 298 may be oriented at an angle θ relative to the longitudinal axis of adaptor 290. Angle θ may range from about 25 degrees to about 75 degrees relative to the longitudinal axis of adaptor 290, from about 40 degrees to about 65 degrees relative to the longitudinal axis of the adaptor 290, or from about 30 degrees to about 60 degrees relative to the longitudinal axis of adaptor 290. In an exemplary embodiment, the angle θ may be about 45 degrees relative to the longitudinal axis of adaptor 290.

Outermost slanted surface 298 may be configured in various manners for contact with a sclera of a patient's eye. For example, outermost slanted surface 298 may be smooth or polished to minimize abrasion of the sclera. Alternatively, outermost slanted surface 298 may be rough to minimize movement of adaptor 290 relative to the sclera. In some embodiments, outermost slanted surface 298 may include geometric features, such as protruding dimples, indented dimples, waves, other geometric features, or any combination thereof. Additionally, a coating may be applied to outermost slanted surface 298. The coating may be therapeutic, antibacterial, and/or sterilizing. As another example, outermost slanted surface 298 may be formed by overmolding a material on adaptor 290. The overmolded material may be selected, for example, based on its surface properties (e.g., rough, smooth, etc.) or its suitability for surface finishing, such as polishing. Outermost slanted surface 298 may further incorporate various combinations of the aforementioned features, such as a polished surface with geometric features, a rough surface with geometric features, an overmolded material with a coating, etc. While exemplary combinations of features have been described herein, these combinations are not intended to be limiting and other combinations are contemplated.

Adaptor 290 may include visual indications of a position of adaptor 290 and/or of outermost slanted surface 298. For example, outermost slanted surface 298 may be colored differently than other surfaces of adaptor 290 to distinguish outermost slanted surface 298 from the other surfaces. Adaptor 290 may also include visible markings to indicate a position adaptor 290 and/or of outermost slanted surface 298. Such visible markings may include markings of contrasting color, textured markings, or the like on outermost slanted surface 298 and/or on other surfaces of adaptor 290. The visible markings may be applied to adaptor 290 using silk-screening, overmolding, etching, or various other suitable techniques. The visible markings may be of any geometric shape, including circles, ovals, polygons, irregular shapes, or any combination thereof.

Adaptor 290 may include a proximal surface 295 and a distal surface 296. Proximal surface 295 may be a substantially circular surface adjacent to intermediate surface 292 and existing in a plane perpendicular to the longitudinal axis of adaptor 290. Distal surface 296 may also be a substantially circular surface. Distal surface 296 may be adjacent to angled distal surface 294 and exist in a separate plane perpendicular to the longitudinal axis of adaptor 290. Accordingly, proximal surface 295 may be parallel to distal surface 296.

Adaptor 290 may include a needle bore 302 in which needle 12 may be positioned. Needle bore 302 may extend parallel or substantially parallel to the longitudinal axis of adaptor 290. When positioned in needle bore 302, needle 12 may intersect each of proximal surface 295 and distal surface 296. When positioned in the needle bore, distalmost tip 18 of needle 12 may extend a distance C from distal surface 296. A length of distance C may be such that a bevel 18a of distalmost tip 18 may extend from distal surface 296. The length of distance C may further be such that a portion of a shaft of needle 12 proximal to distalmost tip 18 may extend from distal surface 296. Distance C may be, for example, between 200 μm and 1200 μm, between 400 μm and 1000 μm, between 600 μm and 800 μm, or about 700 μm. In some implementations, bevel 18a and outermost slanted surface 298 may be oriented at the same angle relative to the longitudinal axis of the adaptor 290.

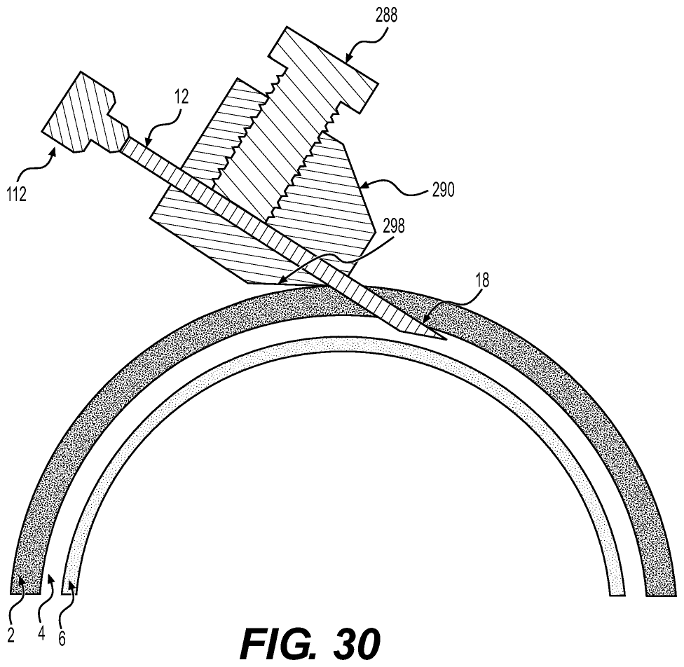

Adaptor 290 may be selectively translatable relative to needle 12 along the longitudinal axis of needle 12. Translation of adaptor 290 may be desirable to adjust the distance C, for example. For use, the adaptor 290 may be fastened to needle 12. Adaptor 290 may be connected to needle 12 by any suitable means, including by a screw, a fastener, a nut, a bolt, or adhesive. As an example, and as shown in FIGS. 28-30, adaptor 290 may be fastened to needle 12 using a screw 288. Screw 288 may be inserted into a threaded bore 304 within adaptor 290. When tightened, screw 288 may exert a force on needle 12 perpendicular to the longitudinal axis of needle 12. The force may result in friction in a longitudinal direction between needle 12 and screw 288 as well as between needle 12 and needle bore 302, thereby preventing adaptor 290 from translating relative to needle 12. If the user wishes to adjust the distance C, e.g. to extend a distance of distalmost tip 18 from distal surface 296, the user may loosen the bolt to thereby allow translation of adaptor 290 relative to needle 12.As shown in FIG. 30, adaptor 290 may be used to guide a trajectory of distalmost tip 18 of needle 12 through sclera 2 into SCS 4. To inject a medicament into SCS 4, a user may, for example, penetrate sclera 2 with distalmost tip 18 and insert needle 12 through sclera 2. The user may angle needle 12 such that outermost slanted surface 298 is oriented parallel to a plane tangent to an outer surface of sclera 2. The user may then continue to insert needle 12 until the outermost slanted surface 298 contacts the surface of sclera 2. In an exemplary method in which outermost slanted surface 298 is a planar surface, the user may insert needle 12 until outermost slanted surface 298 is tangent with the surface of sclera 2. In an exemplary method in which outermost slanted surface 298 is a convex surface, the user may insert needle 12 until outermost slanted surface 298 mates with the surface of sclera 2. When outermost slanted surface 298 contacts sclera 2, needle 12 may be prevented from being inserted further and may be prevented from potentially penetrating choroid 6.

In some implementations, the user may be able to adjust the distance C to a desired length by translating adaptor 290 along needle 12. When the user has adjusted distance C and/or angle θ as desired, the user may use adaptor 290 to guide a trajectory of needle 12 into SCS 4 such that it penetrates sclera 2 at a substantially predetermined depth. Thereby, the user may be able to inject the medicament into the suprachoroidal space 4 with relative accuracy without penetrating choroid 6.

As shown in FIGS. 28-30, adaptor 290 may be positioned about needle 12. Adaptor 290 may alternatively be attached to either or both of hub 112 and a medicament container (e.g. a syringe) connected to needle 12. For example, adaptor 290 may be attached to hub 112 in a similar manner as external shaft 280 as shown in FIGS. 26A and 26B. Moreover, adaptor 290 may be spring-loaded such that a spring urges adaptor 290 toward distalmost tip 18. In use, the user may place adaptor 290 against the patient's sclera and exert a force sufficient to depress the spring, thereby exposing needle 12. The spring may be configured to control a depth of penetration of needle 12 into the patient's eye.

Adaptor 290 may be made of any suitable metal, polymer, and/or combination of metals and/or polymers. Exemplary metallic materials may include stainless steel, nitinol, titanium, and/or alloys of these metals. Exemplary polymeric materials may include polyetheretherketone (PEEK), polyimide, and polyethersulfone (PES). In some examples, adaptor 290 may be made of a rigid material, semi-rigid material, or flexible material. Adaptor 290 may further be formed of any biocompatible material that may be sterilized. In some examples, adaptor 290 may be made of a transparent material to permit easier identification of, and/or navigation relative to, blood vessels in a patient's eye.

It is to be understood that dimensions of adaptor 290 are not intended to be limited and indeed may vary. For example, a length of adaptor 290 (i.e. a distance between proximal surface 295 and distal surface 296) may vary to accommodate needles of different lengths. Also, a diameter of needle bore 302 may vary to accommodate needles having different diameters. Further, diameters of proximal surface 295 and/or distal surface 296 may vary.

As described herein, adaptor 290 may be useful for reducing human error in ocular injection procedures. In addition to being useful for injections into the suprachoroidal space, adaptor 290 may be useful for injections into other spaces in the eye, such as the subretinal space. Current methods for subretinal drug delivery may be invasive and may further require surgery. Surgical procedures for subretinal drug delivery may involve creating tears on the retinal surface and/or full vitrectomies in order to allow for a cannula to access the subretinal space. Alternatively, adaptor 290 may allow access to the subretinal space through the sclera, thereby decreasing the invasiveness of the procedure. Using eye imaging techniques such as optical coherence tomography (OCT) and/or ultrasound, an accurate distance between the surface of the sclera and the subretinal space may be calculated. A distance between distalmost tip 18 of needle 12 and distal surface 296 or outermost slanted surface 298 of adaptor 290 may be configured to match the distance between the sclera and the subretinal space. In such a configuration, adaptor 290 may prevent needle 12 from extending beyond the subretinal space into the vitreous. Outermost slanted surface 298 may also control an angle at which the subretinal injection is performed.

Adaptor 290 may be formed by any suitable manufacturing process, including but not limited to milling, CNC machining, polymer casting, rotational molding, vacuum forming, injection molding, extrusion, blow molding, or any combination thereof.

The various devices and components described herein may be provided in a kit for practicing one or more of the methods described herein. For example, a syringe, a needle, an adaptor, and an amount of ophthalmic drug may be provided in a blister pack. Each of the syringe, the needle, the adaptor, and the ophthalmic drug may be sealed within the blister pack after being sterilized. In some embodiments, a kit may include multiple adaptors. The multiple adaptors may have varying dimensions such that a user may select an adaptor best suited to a patient's anatomy and/or to control a penetration angle or depth of the needle. The multiple adaptors may also be formed from varying materials such that a user may choose an adaptor having an appropriate material for a particular procedure and/or patient. In some embodiments, the syringe may contain the ophthalmic drug. A nominal maximum fill volume of the syringe may be between about 0.5 mL and about 1.0 mL. In various methods described herein, a volume of the medicament, e.g., an ophthalmic drug, delivered to the patient may range from about 50 uL to about 500 uL.

Various drugs and formulations of drugs may be used with the embodiments of the present disclosure. As one example, embodiments described herein may be used to inject a drug in delayed-release pellet form. The drug may be released from the pellets when the pellets are hydrated, which may be achieved either by exposure of the pellets to fluids of the eye, by injecting a separate hydrating fluid, or by a combination of the foregoing. The separate hydrating fluid, such as saline, may be injected either before, after, or simultaneously with the pellets. As another example, embodiments described herein may be used to inject multiple substances in sequence. A first substance may be injected to expand a target space of the eye, such as the suprachoroidal space, and a second substance may subsequently be injected into the expanded suprachoroidal space. The first substance may be, for example, saline and the second substance may be, for example, a drug in a viscous gel form. As still another example, a sponge-like material may first be injected or inserted into a target space of the eye. The sponge-like material may be configured to release a drug over time. The sponge-like material may further be refilled or re-soaked with the drug by subsequent injections of the drug.

Embodiments of the present disclosure may further include additional features to improve accuracy of injections. As one example, embodiments described herein may include a light, such as an LED light, configured to illuminate an injection site. As another example, embodiments described herein may include a needle formed in whole or in part of a magnetic material or otherwise including a magnetic element. A magnet positioned externally to the patient's eye, e.g. held by the user, may be used to guide the needle to the target injection site. The magnet may further be used to pull, i.e. exert a magnetic force, on the needle to prevent the needle from penetrating beyond a desired depth. As yet another example, embodiments described herein may include a mechanism configured to sense an angle of the needle relative to the eye. The mechanism may include a sensor that may be calibrated according to a thickness of the patient's sclera. The thickness of the sclera may be measured using optical coherence topography (OCT), ultrasound, or any other suitable technique. The mechanism may be configured to provide feedback to the user such that if the needle is oriented at an appropriate angle relative to the eye, the user may be alerted to continue advancing the needle. If the needle is not oriented at an appropriate angle relative to the eye, the user may be alerted to cease advancing the needle and adjust the orientation of the needle.

In embodiments of the present disclosure, needle 12 may be a first needle and the devices, apparatus, and/or kits disclosed herein may include a second needle. The first needle and the second needle may be interchangeable. Accordingly, needle 12 maybe be replaceable.

Listed below are further illustrative embodiments according to the present disclosure:

(1) A system for delivering medicament to a suprachoroidal space of an eye, the system comprising: a needle having a passage therethrough and a sharp distalmost tip; and an apparatus configured to manipulate a sclera to facilitate delivery of the medicament to the suprachoroidal space of the eye.

(2) The system of (1), wherein the needle is configured to deliver the medicament to the suprachoroidal space of the eye.

(3) The system of (1), wherein the sharp distalmost tip includes a plurality of openings.

(4) The system of (3), wherein the openings include a circular configuration.

(5) The system of (3), wherein the openings include slots.

(6) The system of (3), wherein the plurality of openings are present on at least a portion of a circumference and a length of the sharp distalmost tip.

(7) The system of (1), wherein the apparatus is configured to deliver the medicament to the suprachoroidal space of the eye.

(8) The system of (1), wherein the apparatus is disposed within the passage of the needle and longitudinally translatable relative to the needle.

(9) The system of (8), wherein the apparatus includes a tubular shaft having a distal end, wherein the tubular shaft comprises a rigid material, a semi-rigid material, or a flexible material.

(10) The system of (9), wherein the distal end includes an atraumatic distal tip.

(11) The system of (10), wherein the atraumatic distal tip includes a plurality of openings.

(12) The system of (11), wherein the openings include a circular configuration.

(13) The system of (11), wherein the openings include slots.

(14) The system of (11), wherein the plurality of openings are present on at least a portion of a circumference and a length of the atraumatic distal tip.

(15) The system of (9), wherein the distal end includes an expandable member.

(16) The system of (15), wherein the expandable member includes a stent.

(17) The system of (10), wherein the tubular shaft includes an expandable portion.

(18) The system of (17), wherein the expandable portion includes a pair of curved arms positioned proximally of the atraumatic distal tip.

(19) The system of (9), wherein the tubular shaft includes a cross-sectional dimension less than a cross-sectional dimension of the passage.

(20) The system of (9), wherein an outer surface of the tubular shaft includes one or more channels.

(21) The system of (9), wherein the distal end of the tubular shaft includes at least two legs configured to selectively diverge when the distal end of tubular shaft is deployed from the passage of the needle.

(22) The system of (1), wherein the needle is curved.

(23) The system of (1), wherein the needle is U-shaped.

(24) The system of (1), wherein the apparatus comprises a planar surface having a projection for deforming one or more of the sclera or choroid.

(25) The system of (24), wherein the projection is a rounded surface.

(26) The system of (1), wherein the apparatus includes a chamber configured to draw a portion of the sclera therein.

(27) The system of (26), wherein the apparatus is configured to apply a suction to the sclera.

(28) The system of (27), wherein the needle is disposed within the chamber.

(29) The system of (27), wherein the chamber includes a stop configured to limit proximal advancement of the sclera into the chamber.

(30) The system of (29), wherein the stop is configured to surround the needle.

(31) The system of (29), wherein the stop includes a plurality of extensions extending from a sidewall of the chamber towards a center of the chamber.

(32) The system of (29), wherein the stop includes a plurality of openings configured to facilitate application of suction to the sclera.

(33) The system of (26), wherein the chamber includes a circular cross-section configuration.

(34) The system of (26), wherein the chamber includes a semi-circular cross-sectional configuration.

(35) The system of (1), wherein the needle includes a bend positioned proximally of the sharp distalmost tip.

(36) The system of (1), wherein the needle includes an outer surface having a plurality of geometric features configured to provide tactile feedback to a user.

(37) The system of (9), wherein the distal end includes an anchor.

(38) The system of (37), wherein the anchor has a curved, planar, or atraumatic shape.

(39) An apparatus for manipulating a sclera to facilitate delivery of a medicament to a suprachoroidal space of an eye, the apparatus comprising: a tubular shaft having a distal end, wherein the tubular shaft comprises a rigid, semi-rigid, or flexible material; and a needle having a passage therethrough and a sharp distalmost tip, wherein the apparatus is disposed within the needle.

(40) The apparatus of (39), wherein the needle is configured to deliver the medicament to the suprachoroidal space of the eye.

(41) The apparatus of (39), wherein the sharp distalmost tip includes a plurality of openings.

(42) The apparatus of (41), wherein the openings include a circular configuration.

(43) The apparatus of (41), wherein the openings include slots.

(44) The apparatus of (41), wherein the plurality of openings are present on at least a portion of a circumference and a length of the sharp distalmost tip.

(45) The apparatus of (39), wherein the apparatus is configured to deliver the medicament to the suprachoroidal space of the eye.

(46) The apparatus of (39), wherein the apparatus is longitudinally translatable relative to the needle.

(47) The apparatus of (39), wherein the distal end includes an atraumatic distal tip.

(48) The apparatus of (47), wherein the atraumatic distal tip includes a plurality of openings.

(49) The apparatus of (48), wherein the openings include a circular configuration.

(50) The apparatus of (48), wherein the openings include slots.

(51) The apparatus of (48), wherein the plurality of openings are present on at least a portion of a circumference and a length of the atraumatic distal tip.

(52) The apparatus of (39), wherein the distal end includes an expandable member.

(53) The apparatus of (52), wherein the expandable member includes a stent.

(54) The apparatus of (39), wherein the tubular shaft includes an expandable portion.

(55) The apparatus of (54), wherein the expandable portion includes a pair of curved arms positioned proximally of the distal end.

(56) The apparatus of (39), wherein the tubular shaft includes a cross-sectional dimension less than a cross-sectional dimension of the passage.

(57) The apparatus of (39), wherein an outer surface of the tubular shaft includes one or more channels.

(58) The apparatus of (39), wherein the distal end of the tubular shaft includes at least two legs configured to selectively diverge when the distal end of tubular shaft is deployed from the passage of the needle.

(59) The apparatus of (39), wherein the needle is curved.

(60) The apparatus of (39), wherein the distal end includes an anchor.

(61) The apparatus of (60), wherein the anchor has a curved, planar, or atraumatic shape.

(62) An apparatus for manipulating a sclera to facilitate delivery of a medicament to a suprachoroidal space of an eye, the apparatus comprising a planar surface having a projection for deforming one or more of the sclera or choroid.

(63) The apparatus of (62), wherein the projection is a rounded surface.

(64) An apparatus for manipulating a sclera to facilitate delivery of a medicament to a suprachoroidal space of an eye, the apparatus comprising a chamber configured to draw a portion of the sclera therein.

(65) The apparatus of (64), wherein the apparatus is configured to apply a suction to the sclera.

(66) The apparatus of (64), wherein a needle is disposed within the chamber.

(67) The apparatus of (66), wherein the chamber includes a stop configured to proximal advancement of the sclera into the chamber.

(68) The apparatus of (67), wherein the stop is configured to surround the needle.

(69) The apparatus of (67), wherein the stop includes a plurality of extensions extending from a sidewall of the chamber towards a center of the chamber.

(70) The apparatus of (67), wherein the stop includes a plurality of openings configured to facilitate application of suction to the sclera.

(71) The apparatus of (67), wherein the chamber includes a circular cross-section configuration.

(72) The apparatus of (67), wherein the chamber includes a semi-circular cross-sectional configuration.

(73) An apparatus for manipulating a sclera to facilitate delivery of a medicament to a suprachoroidal space of an eye, the apparatus comprising a needle tubing having a cylindrical shape and a serrated needle tip.

(74) The apparatus of (73), wherein the serrated needle tip oscillates to cut through a portion of the sclera.

(75) An apparatus for manipulating a sclera to facilitate delivery of a medicament to a suprachoroidal space of an eye, the apparatus comprising: a needle with a sharp distalmost tip; a needle hub connected to a proximal end of the needle; and a housing surrounding the needle hub and extending from a proximal end of the needle hub.

(76) The apparatus of (75), wherein the housing is cylindrical shaped.

(77) The apparatus of (75), wherein the housing comprises an additional component chosen from a syringe, a spring, a piston, a plunger rod, an indicator, a feedback mechanism, or combinations thereof.

(78) The apparatus of (75), further comprising a shaft surrounding a portion of the needle and extending from a distal end of the needle hub.

(79) The apparatus of (78), wherein a distal end of the shaft is angled, allowing for insertion of the needle at an angle.

(80) A method for delivering a medicament to a suprachoroidal space of an eye, the method comprising: manipulating one of a sclera and a choroid layer of the eye to increase a dimension of the suprachoroidal space; advancing a distal end of a medicament delivery device to the suprachoroidal space; positioning a distalmost tip of the medicament delivery device in the suprachoroidal space; and delivering a volume of the medicament to the suprachoroidal space.

(81) The method of (80), wherein advancing the distal end of the medicament delivery device to the suprachoroidal space includes penetrating the sclera.

(82) The method of (80), wherein positioning the distalmost tip of the medicament delivery device includes disposing the distalmost tip into the suprachoroidal space without contacting the choroid.

(83) The method of (80), wherein positioning the distalmost tip of the medicament delivery device includes disposing the distalmost tip into the suprachoroidal space without piercing an outermost surface of the choroid.

(84) The method of (80), wherein positioning the distalmost tip of the medicament includes disposing the distalmost tip in the suprachoroidal space without penetrating a thickness of the choroid.

(85) The method of (80), wherein a volume of the medicament delivered to the suprachoroidal space is approximately 50 uL to 500 uL.

(86) The method of (80), wherein delivery of the volume of the medicament to the suprachoroidal space is pressure controlled.

(87) The method of (80), wherein manipulating one of the sclera and the choroid layer includes rotating the medicament delivery device.

(88) The method of (80), wherein manipulating one of the sclera and choroid layer includes pulling the sclera layer to increase the dimension of the suprachoroidal space.

(89) The method of (80), wherein delivering the volume of the medicament to the suprachoroidal space include delivering the volume from the distalmost tip of the medicament delivery device.

(90) The method of (80), wherein delivering a volume of the medicament to the suprachoroidal space includes delivering the medicament from a location proximally of the distalmost tip of the medicament delivery device.

(91) An apparatus to facilitate directed delivery of a medicament into a human organ of a patient, the apparatus comprising: a container for a medicament fluidly connected to a needle, the needle comprising a needle shaft and a sharp distalmost tip with a bevel, wherein the needle is connected to a distal end of the container, and an adaptor surrounding a portion of the needle shaft along its longitudinal axis, but not including the sharp distalmost tip of the needle, the adaptor including an outermost slanted surface configured to direct a trajectory of the sharp distalmost tip to a pre-determined depth and location within the human organ, wherein the outermost slanted surface faces an identical direction as the bevel of the sharp distalmost tip, wherein an angle of the outermost slanted surface dictates the trajectory of the needle, and wherein a length of the needle extending from the outermost slanted surface determines the depth and location of the medicament delivery.

(92) The apparatus of (91), wherein the sharp distalmost tip is a portion of the needle extending from a distal end of the adaptor.

(93) The apparatus of (91), further comprising a needle hub shaft connected to a proximal end of the needle, such as a staked needle.

(94) The apparatus of (91), further comprising a hub disposed between the container and the needle.

(95) The apparatus of (94), wherein the needle is removably connected to the hub.

(96) The apparatus of (95), wherein the needle is a first needle, and the apparatus further comprises a second needle.

(97) The apparatus of (96), wherein the first needle and the second needle are interchangeable.

(98) The apparatus of (91), wherein the needle is replaceable.

(99) The apparatus of (91), wherein the angle ranges from about 25 degrees to about 75 degrees.

(100) The apparatus of (91), wherein the angle ranges from about 40 degrees to about 60 degrees.

(101) The apparatus of (91), wherein the angle is about 45 degrees.

(102) The apparatus of (91), wherein the adaptor is connected to a portion of the needle shaft via a fastener or screw.

(103) The apparatus of (102), wherein the adaptor is translatable relative to an axial path of the needle shaft.

(104) The apparatus of (91), wherein the adaptor is attached to the needle shaft via an adhesive.

(105) The apparatus of (91), wherein the adaptor includes: a proximal end having a surface extending in a first plane perpendicular to the needle; an angled distal side; an intermediate surface extending between the proximal end and the angled distal side, wherein the intermediate surface extends in a second plane perpendicular to the first plane; and a distal end, wherein the distal end includes a substantially flat surface extending in a third plane parallel to the first plane.

(106) The apparatus of (91), wherein at least a portion of the adaptor includes a substantially cylindrically shaped cross-section.

(107) The apparatus of (91), wherein the outermost slanted surface is angled relative to a longitudinal axis of the adaptor.

(108) The apparatus of (91), wherein the sharp distalmost tip of the needle has a length ranging from about 600 μm to about 800 μm.

(109) The apparatus of (91), wherein the outermost slanted surface is a planar surface.

(110) The apparatus of (91), wherein the outermost slanted surface is a convex surface configured to mate with an outer surface of an eye.

(111) A system for delivering medicament to an ocular space of a patient, the system comprising: a syringe with a nominal maximum fill volume of between about 0.5 mL and about 1.0 mL; a needle comprising a needle shaft and a sharp distalmost tip with a bevel; and an adaptor surrounding a portion of the needle shaft along its longitudinal axis, the adaptor including an outermost slanted surface angled relative to a longitudinal axis of the adaptor and wherein the adaptor is configured to direct a trajectory of the sharp distalmost tip to a pre-determined ocular depth and location; wherein the syringe, needle, and adaptor are sterilized and contained in a blister pack.

(112) The system of (111), wherein the adaptor is configured to limit advancement of the sharp distalmost tip into a suprachoroidal space of an eye.

(113) The system of (111), wherein the sharp distalmost tip is a portion of the needle extending from the outermost slanted surface.

(114) The system of (111), wherein an angle of the outermost slanted surface ranges from about 25 degrees to about 75 degrees.

(115) The system of (111), wherein an angle of the outermost slanted surface ranges from about 40 degrees to about 60 degrees.

(116) The system of (111), wherein an angle of the outermost slanted surface is about 45 degrees.

(117) The system of (111), wherein the sharp distalmost tip of the needle has a length ranging from about 600 μm to about 800 μm.

(118) A kit for treating a patient suffering from an ocular disease, the kit comprising: a syringe with a nominal maximum fill volume of between about 0.5 ml and about 1.0 ml; a needle having a needle shaft and a sharp distalmost tip; an adaptor surrounding a portion of the needle shaft along its longitudinal axis, but not including the sharp distalmost tip of the needle, the adaptor including an outermost slanted surface configured to direct a trajectory of the sharp distalmost tip to a pre-determined ocular depth and location; and an ophthalmic drug.

(119) A kit for treating a patient suffering from an ocular disease, the kit comprising: a syringe pre-filled with an ophthalmic drug, wherein a volume of the ophthalmic drug ranges between about 0.5 ml and about 1.0 ml; a needle having a needle shaft, a passage therethrough, and a sharp distalmost tip; and an adaptor surrounding a portion of the needle shaft along a longitudinal axis of the needle shaft, the portion excluding the sharp distalmost tip of the needle, the adaptor including an outermost slanted surface configured to direct a trajectory of the sharp distalmost tip to a predetermined ocular depth and location.

(120) A method of delivering a medicament to an eye of a patient, the method comprising: positioning a distalmost tip of a medicament delivery device in a suprachoroidal space of the eye at a predetermined ocular depth and location, wherein the medicament delivery device comprises: a container for a medicament fluidly connected to a needle, the needle comprising a needle shaft and a sharp distalmost tip with a bevel, wherein the needle is connected to a distal end of the container, and an adaptor surrounding a portion of the needle shaft along its longitudinal axis, the portion not including the sharp distalmost tip of the needle, the adaptor including an outermost slanted surface configured to direct a trajectory of the sharp distalmost tip to a pre-determined ocular depth and location; and delivering a volume of the medicament to the suprachoroidal space.

(121) A method for delivering a medicament to a suprachoroidal space of an eye using a medicament device, the medicament device comprising: a container for a medicament fluidly connected to a needle, the needle comprising a needle shaft and a sharp distalmost tip with a bevel; and an adaptor surrounding a portion of the needle shaft along its longitudinal axis, the adaptor including an outermost slanted surface angled with respect to the longitudinal axis; the method comprising: penetrating a sclera of the eye with the sharp distalmost tip; inserting the needle through the sclera into the suprachoroidal space until the outermost slanted surface contacts the sclera; and upon the outermost slanted surface contacting the sclera, delivering a volume of the medicament to the suprachoroidal space.

(122) A method for delivering a medicament to a suprachoroidal space of an eye, the method comprising: manipulating one of a sclera or a choroid layer of the eye to increase a dimension of the suprachoroidal space; advancing a distal end of a medicament delivery device to the suprachoroidal space; positioning a distalmost tip of the medicament delivery device in the suprachoroidal space; and delivering a volume of the medicament to the suprachoroidal space.

(123) The method of (122), wherein advancing the distal end of the medicament delivery device to the suprachoroidal space includes penetrating the sclera.

(124) The method of (122), wherein positioning the distalmost tip of the medicament delivery device includes disposing the distalmost tip into the suprachoroidal space without contacting the choroid.

(125) The method of (122), wherein positioning the distalmost tip of the medicament delivery device includes disposing the distalmost tip into the suprachoroidal space without piercing an outermost surface of the choroid.

(126) The method of (122), wherein positioning the distalmost tip of the medicament includes disposing the distalmost tip in the suprachoroidal space without penetrating a thickness of the choroid.

(127) The method of (122), wherein a volume of the medicament delivered to the suprachoroidal space is approximately 50 uL to 500 uL.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

What is claimed is:

1. An apparatus for manipulating a sclera to facilitate delivery of a medicament to a suprachoroidal space of an eye, the apparatus comprising:
a needle with a sharp distalmost tip;
a needle hub connected to a proximal end of the needle;
a housing surrounding the needle hub and extending from a proximal end of the needle hub; and
an adaptor surrounding a portion of the needle.

2. The apparatus of claim 1, wherein a distal end of the adaptor is angled, allowing for insertion of the needle at a desired angle.

3. The apparatus of claim 1, wherein the adaptor is a shaft attached to the needle hub.

4. The apparatus of claim 3, wherein the adaptor extends from a distal end of the needle hub toward the sharp distalmost tip.

5. The apparatus of claim 3, wherein a distal end of the adaptor is offset a first distance from the distalmost tip of the needle.

6. The apparatus of claim 5, wherein the adaptor is configured to allow the first distance to be adjusted.

7. The apparatus of claim 1, wherein the adaptor includes an angled edge configured to enable needle insertion at a desired angle.

8. The apparatus of claim 1, wherein the sharp distalmost tip is a portion of the needle extending from a distal end of the adaptor.

9. An apparatus to facilitate directed delivery of a medicament into a human organ of a patient, the apparatus comprising:
a container for a medicament fluidly connected to a needle, the needle comprising a needle shaft and a sharp distalmost tip with a bevel, wherein the needle is connected to a distal end of the container, and
an adaptor surrounding a portion of the needle shaft along its longitudinal axis, the portion not including the sharp distalmost tip of the needle, the adaptor including an outermost slanted surface configured to direct a trajectory of the sharp distalmost tip to a pre-determined depth and location within the human organ,
wherein the outermost slanted surface faces an identical direction as the bevel of the sharp distalmost tip,
wherein an angle of the outermost slanted surface dictates the trajectory of the needle, and
wherein a length of the needle extending from the outermost slanted surface determines the depth and location of the medicament delivery.

10. The apparatus of claim 9, wherein the sharp distalmost tip is a portion of the needle extending from a distal end of the adaptor.

11. The apparatus of claim 9, further comprising a needle hub shaft connected to a proximal end of the needle, such as a staked needle.

12. The apparatus of claim 9, wherein the angle ranges from about 25 degrees to about 75 degrees.

13. The apparatus of claim 9, wherein the angle ranges from about 40 degrees to about 60 degrees.

14. The apparatus of claim 9, wherein the adaptor is connected to a portion of the needle shaft via a fastener or screw.

15. The apparatus of claim 14, wherein the adaptor is translatable relative to a longitudinal axis of the needle shaft.

16. The apparatus of claim 9, wherein the adaptor includes:
a proximal end having a surface extending in a first plane perpendicular to the needle;
an angled distal side;
an intermediate surface extending between the proximal end and the angled distal side, wherein the intermediate surface extends in a second plane perpendicular to the first plane; and
a distal end, wherein the distal end includes a substantially flat surface extending in a third plane parallel to the first plane.

17. The apparatus of claim 9, wherein the outermost slanted surface is angled relative to a longitudinal axis of the adaptor.

18. The apparatus of claim 9, wherein the outermost slanted surface is a convex surface configured to mate with an outer surface of an eye.

19. A method of delivering a medicament to an eye of a patient, the method comprising:
positioning a distalmost tip of a medicament delivery device in a suprachoroidal space of the eye at a predetermined ocular depth and location, wherein the medicament delivery device comprises:
a container for a medicament fluidly connected to a needle, the needle comprising a needle shaft and a sharp distalmost tip with a bevel, wherein the needle is connected to a distal end of the container, and
an adaptor surrounding a portion of the needle shaft along its longitudinal axis, the portion not including the sharp distalmost tip of the needle, the adaptor including an angled surface configured to direct a trajectory of the sharp distalmost tip to a pre-determined ocular depth and location; and
delivering a volume of the medicament to the suprachoroidal space.

20. The method of claim 19 wherein positioning the distalmost tip of the medicament delivery device in the suprachoroidal space includes penetrating a sclera.

21. The method of claim 20, wherein positioning the distalmost tip of the medicament delivery device includes disposing the distalmost tip into the suprachoroidal space without piercing an outermost surface of a choroid.

22. The method of claim 19, wherein a volume of the medicament delivered to the suprachoroidal space is approximately 50 uL to 500 uL.

23. A method for delivering a medicament to a suprachoroidal space of an eye using a medicament device, the medicament device comprising:
a container for a medicament fluidly connected to a needle, the needle including a needle shaft and a sharp distalmost tip with a bevel; and
an adaptor surrounding a portion of the needle shaft along its longitudinal axis, the adaptor including an angled surface sloped relative to the longitudinal axis;
the method comprising:
penetrating a sclera of the eye with the sharp distalmost tip;
inserting the needle through the sclera into the suprachoroidal space until the angled surface comes in contact with the sclera; and
upon the angled surface contacting the sclera, delivering a volume of the medicament to the suprachoroidal space.

24. The method of claim 23, wherein inserting the needle through the sclera into the suprachoroidal space includes disposing the distalmost tip into the suprachoroidal space without piercing an outermost surface of a choroid.

25. The method of claim 23, further comprising:
adjusting a position of the adaptor relative to the longitudinal axis such that the adaptor prevents the distalmost tip from piercing an outermost surface of a choroid.

26. A kit for treating a patient suffering from an ocular disease, the kit comprising:
a syringe with a nominal maximum fill volume of between about 0.5 ml and about 1.0 ml;
a needle having a needle shaft and a sharp distalmost tip;
a first adaptor configured to surround a portion of the needle shaft along its longitudinal axis, the portion not including the sharp distalmost tip of the needle, the first adaptor configured to direct a trajectory of the sharp distalmost tip to a first pre-determined ocular depth and location; and
an ophthalmic drug.

27. The kit of claim 26, further comprising:
a second adaptor having dimensions differing from the first adaptor, the second adaptor configured to direct the trajectory of the sharp distalmost tip to a second pre-determined ocular depth and location, the second pre-determined ocular depth and location differing from the first pre-determined ocular depth and location.

28. The kit of claim 26, wherein the syringe, the needle, the first adaptor, and the ophthalmic drug are sealed within a blister pack.
